SEQUENCE LISTING (15 sequences)

SEQ ID NO: 1 - protein, 5 aa, synthetic construct
DSVMN

SEQ ID NO: 2 - protein, 17 aa, synthetic construct
WIDPEYGRTDVAEKFQG

SEQ ID NO: 3 - protein, 11 aa, synthetic construct
TKYNSGYGFPY

SEQ ID NO: 4 - protein, 11 aa, synthetic construct
KASQNVDSDVD

SEQ ID NO: 5 - protein, 7 aa, synthetic construct
KASNDYT

SEQ ID NO: 6 - protein, 9 aa, synthetic construct
MQSNTHPRT

SEQ ID NO: 7 - protein, 120 aa, synthetic construct
EVQLVQSGAEVKKPGATVKISCKVSGYTFTDSVMNWVQQAPGKGLEWMGWIDPEYGRTDVAEKFQGRVTITADTSTDTAYMELSSLRSEDTAVYYCARTKYNSGYGFPYWGQGTTVTVSS

SEQ ID NO: 8 - protein, 108 aa, synthetic construct
DIQMTQSPSSLSASVGDRVTITCKASQNVDSDVDWYQQKPGKAPKLLIYKASNDYTGVPSRFSGSGSGTDFTFTISSLQPEDIATYYCMQSNTHPRTFGGGTKVEIKR

SEQ ID NO: 9 - protein, 447 aa, synthetic construct
EVQLVQSGAEVKKPGATVKISCKVSGYTFTDSVMNWVQQAPGKGLEWMGWIDPEYGRTDVAEKFQGRVTITADTSTDTAYMELSSLRSEDTAVYYCARTKYNSGYGFPYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQMTQSPSSLSASVGDRVTITCKASQNVDSDVDWYQQKPGKAPKLLIYKASNDYTGVPSRFSGSGSGTDFTFTISSLQPEDIATYYCMQSNTHPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 165 aa, Homo sapiens [sequence omitted]

SEQ ID NO: 12 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVAETPLMKEDSILAVRKYFQRITLYLKEKKYSPCAWEVVRDEIMRSFSLSTNLQESLRSKE

SEQ ID NO: 13 - protein, 612 aa, synthetic construct
EVQLVQSGAEVKKPGATVKISCKVSGYTFTDSVMNWVQQAPGKGLEWMGWIDPEYGRTDVAEKFQGRVTITADTSTDTAYMELSSLRSEDTAVYYCARTKYNSGYGFPYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVAETPLMKEDSILAVRKYFQRITLYLKEKKYSPCAWEVVRDEIMRSFSLSTNLQESLRSKE

SEQ ID NO: 14 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0265401 A1
(43) Pub. Date: Sep. 10, 2026

(54) DOSING OF CD38-BINDING FUSION PROTEIN

(71) Applicant: Takeda Pharmaceutical Company Limited, Osaka-shi, Osaka (JP)

(72) Inventors: Sabrina Collins, Cambridge, MA (US); Xavier Parot, Cambridge, MA (US); Shuang Wu Li, Cambridge, MA (US); Neeraj Gupta, Cambridge, MA (US); Shining Wang, Cambridge, MA (US); Tian Chen, Cambridge, MA (US); Hui Yang, Cambridge, MA (US); Yuyin Liu, Cambridge, MA (US); Ignacio Garcia Ribas, Cambridge, MA (US); Ryan Larson, Cambridge, MA (US)

(73) Assignee: Takeda Pharmaceutical Company Limited, Osaka-shi, Osaka (JP)

(21) Appl. No.: 18/869,419

(22) PCT Filed: May 26, 2023
(86) PCT No.: PCT/IB2023/000322

§ 371 Date: Nov. 26, 2024

Related U.S. Application Data
(60) Provisional application No. 63/346,784, filed on May 27, 2022.
(60) Provisional application No. 63/488,019, filed on Mar. 2, 2023.

(51) Int. Cl. C07K 16/28; A61K 31/167; A61K 39/00; A61K 45/06; A61K 47/18; A61K 47/22; A61K 47/26; A61P 35/00; C07K 14/56

(52) U.S. Cl. CPC C07K 16/2896; A61K 31/167; A61K 45/06; A61K 47/183; A61K 47/22; A61K 47/26; A61P 35/00; C07K 14/56; C07K 16/2818; A61K 2039/545; C07K 2317/52; C07K 2317/565; C07K 2319/00

(57) ABSTRACT

Disclosed herein are methods of treating cancer (e.g., hematologic or solid tumor) with a CD38-binding fusion protein, and optionally in combination with an immune checkpoint inhibitor (e.g., a PD-1 inhibitor such as pembrolizumab).

This application claims the benefit under 35 U.S.C. § 119(e) of U.S. provisional application No. 63/346,784, titled “DOSING OF ANTI-CD38 FUSION PROTEIN,” filed May 27, 2022 and U.S. provisional application No. 63/488,019, titled “DOSING OF ANTI-CD38 FUSION PROTEIN,” filed Mar. 2, 2023 each of which is incorporated by reference herein in its entirety.

### BACKGROUND

CD38 is a 46 kDa type II transmembrane glycoprotein. It has a short N-terminal cytoplasmic tail of 20 amino acids, a single transmembrane helix and a long extracellular domain of 256 amino acids. It is expressed on the surface of many immune cells including CD4 and CD8 positive T cells, B cells, NK cells, monocytes, plasma cells, and on a significant proportion of normal bone marrow precursor cells. CD38 is expressed at high levels on various types of cancer cells, e.g., multiple myeloma cells, in most cases of T- and B-lineage acute lymphoblastic leukemias, some acute myelocytic leukemias, follicular center cell lymphomas and T lymphoblastic lymphomas. CD38 is also expressed on B-lineage chronic lymphoblastic leukemia (B-CLL) cells. Antibodies that target CD38 have been used in the treatment of CD38-expressing cancers and hematological malignancies.

Interferons, and in particular IFN-alpha, are able to increase apoptosis and decrease proliferation of certain cancer cells. IFN-alpha has been approved by the FDA for the treatment of several cancers including melanoma, renal cell carcinoma, B cell lymphoma, multiple myeloma, chronic myelogenous leukemia (CML) and hairy cell leukemia. In general, IFN may be targeted to cancer cells, for example, by linking it with a targeting antibody or targeting fragment thereof.

Fusion proteins containing anti-CD38 antibodies fused to IFN-alpha and their use in treating cancer have been described.

### SUMMARY

The present disclosure, in some aspects, relates to methods of treating cancer, the method comprising administering to a subject in need thereof a composition comprising a CD38-binding fusion protein, wherein the CD38-binding fusion protein comprises an anti-CD38 antibody fused to an attenuated interferon alpha-2b.

In some embodiments, the CD38-binding fusion protein is administered at 0.1-1.5 mg/kg of the subject. In some embodiments, the CD38-binding fusion protein is administered at 1 mg/kg of the subject. In some embodiments, the CD38-binding fusion protein is administered at 0.75 mg/kg of the subject. In some embodiments, the CD38-binding fusion protein is administered at 0.75-1.5 mg/kg of the subject.

In some embodiments, the method comprises administering to the subject a composition comprising 60-120 mg of the CD38-binding fusion protein (e.g., regardless of the subject's weight). In some embodiments, 80 mg of the CD38-binding fusion protein is administered (e.g., regardless of the subject's weight). In some embodiments, the 120 mg of the CD38-binding fusion protein is administered (e.g., regardless of the subject's weight).

In some embodiments, the CD38-binding fusion protein is administered once every three weeks.

In some embodiments, the anti-CD38 antibody comprises a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, a light chain complementarity determining region 3 (CDR-L3) comprising the amino acid sequence of SEQ ID NO: 6.

In some embodiments, the anti-CD38 antibody comprises a heavy chain variable region comprising the amino acid sequence of SEQ ID NO: 7 and a light chain variable region comprising the amino acid sequence of SEQ ID NO: 8. In some embodiments, the anti-CD38 antibody comprises a human IgG4 constant region. In some embodiments, the human IgG4 constant region comprises a proline at position 228 according to the EU numbering system. In some embodiments, the human IgG4 constant region further comprises a tyrosine at position 252, a threonine at position 254, and a glutamic acid at position 256 of the constant region according to the EU numbering system. In some embodiments, the anti-CD38 antibody comprises a heavy chain comprising the amino acid sequence of SEQ ID NO: 9 and a light chain comprising the amino acid sequence of SEQ ID NO: 10. In some embodiments, the attenuated interferon alpha-2b comprises T106A and A145D mutations relative to an interferon alpha-2b comprising the amino acid sequence of SEQ ID NO: 11. In some embodiments, the attenuated interferon alpha-2b comprises the amino acid sequence of SEQ ID NO: 12. In some embodiments, the attenuated interferon alpha-2b is fused to the C-terminus of the heavy chain. In some embodiments, the CD38-binding fusion protein comprises a heavy chain comprising the amino acid sequence of SEQ ID NO: 13 and a light chain comprising the amino acid sequence of SEQ ID NO: 10.

In some embodiments, the composition further comprises histidine, arginine, sucrose, and polysorbate 80 (PS80). In some embodiments, in the composition, histidine is at a concentration of 50 mM, arginine is at a concentration of 100 mM, sucrose is at a concentration of 50 mg/ml, and PS80 is at a concentration of 0.2 mg/ml. In some embodiments, the composition is at a pH of 6.6.

In some embodiments, the method further comprises administering to the subject an immune checkpoint inhibitor. In some embodiments, the immune checkpoint inhibitor is a PD-1 inhibitor. In some embodiments, the PD-1 inhibitor comprises an anti-PD-1 antibody. In some embodiments, the anti-PD1 antibody is pembrolizumab. In some embodiments, 400 mg of pembrolizumab is administered once every six weeks. In some embodiments, 200 mg of pembrolizumab is administered once every three weeks.

In some embodiments, the cancer is a solid tumor. In some embodiments, the cancer is metastatic. In some embodiments, the cancer is a colorectal cancer, bile duct cancer, bone cancer, gastroesophageal cancer, pancreatic cancer, renal cancer, melanoma, anal cancer, uterine cancer, liver cancer, muscle cancer, breast cancer, bladder cancer, brain cancer, head and neck cancer, ovarian cancer, cervical cancer, prostate cancer, endometrial cancer, or stomach cancer, optionally wherein the cancer is metastatic melanoma, metastatic non-small cell lung cancer, head and neck squamous cell carcinoma, esophageal squamous cell carcinoma, metastatic urothelial carcinoma, gastric or gastroesophageal junction (GEJ) adenocarcinoma, triple-negative breast cancer, high-risk non-muscle invasive bladder cancer, advanced urothelial bladder cancer, Merkel cell carcinoma, and cutaneous squamous cell carcinoma. In some embodiments, the cancer is a CD38-expressing cancer. In some embodiments, the cancer does not express CD38 (e.g., does not express CD38 as measured experimentally). In some embodiments, the cancer is melanoma. In some embodiments, the subject is human.

Further provided herein are methods of treating cancer, the method comprising administering to a subject in need thereof a composition comprising a CD38-binding fusion protein, wherein the CD38-binding fusion protein comprises an anti-CD38 antibody fused to an attenuated interferon alpha-2b, wherein the CD38-binding fusion protein is administered at 0.1-1.5 mg/kg of the subject once every three weeks, and wherein the subject is receiving or has received treatment with an immune checkpoint inhibitor.

Further provided herein are methods of treating cancer, the method comprising administering to a subject in need thereof a composition comprising an immune checkpoint inhibitor, wherein the subject is receiving or has received treatment with a CD38-binding fusion protein, wherein the CD38-binding fusion protein comprises an anti-CD38 antibody fused to an attenuated interferon alpha-2b, wherein the CD38-binding fusion protein is administered at 0.1-1.5 mg/kg of the subject once every three weeks.

Further provided herein are methods of treating cancer, the method comprising administering to a subject in need thereof a composition comprising a CD38-binding fusion protein, wherein the CD38-binding fusion protein comprises an anti-CD38 antibody fused to an attenuated interferon alpha-2b, wherein 60-120 mg of the CD38-binding fusion protein is administered once every three weeks, and wherein the subject is receiving or has received treatment with an immune checkpoint inhibitor.

Further provided herein are methods of treating cancer, the method comprising administering to a subject in need thereof a composition comprising an immune checkpoint inhibitor, wherein the subject is receiving or has received treatment with a CD38-binding fusion protein, wherein the CD38-binding fusion protein comprises an anti-CD38 antibody fused to an attenuated interferon alpha-2b, wherein 60-120 mg of the CD38-binding fusion protein is administered once every three weeks.

Further provided herein are CD38-binding fusion proteins for use in a method of treating cancer, the method comprising administering to a subject in need thereof the CD38-binding fusion protein at 0.1-1.5 mg/kg of the subject once every three weeks, wherein the CD38-binding fusion protein comprises an anti-CD38 antibody fused to an attenuated interferon alpha-2b.

Further provided herein are CD38-binding fusion proteins for use in a method of treating cancer, the method comprising administering to a subject in need thereof 60-120 mg of the CD38-binding fusion protein once every three weeks, wherein the CD38-binding fusion protein comprises an anti-CD38 antibody fused to an attenuated interferon alpha-2b.

In some embodiments, the subject is further administered an immune checkpoint inhibitor. In some embodiments, the immune checkpoint inhibitor is pembrolizumab. In some embodiments, 400 mg of pembrolizumab is administered once every six weeks. In some embodiments, wherein 200 mg of pembrolizumab is administered once every three weeks.

In some embodiments, the CD38-binding fusion protein comprises a heavy chain comprising the amino acid sequence of SEQ ID NO: 13 and a light chain comprising the amino acid sequence of SEQ ID NO: 10. In some embodiments, the subject is human. In some embodiments, the cancer is a metastatic solid tumor. In some embodiments, the cancer is melanoma.

In some aspects, disclosed herein are methods of treating multiple myeloma, the methods comprising administering to a subject in need thereof a composition comprising a CD38-binding fusion protein, wherein the CD38-binding fusion protein comprises an anti-CD38 antibody fused to an attenuated interferon alpha-2b, wherein the CD38-binding fusion protein is administered at 1.5-3 mg/kg of the subject or wherein 120-240 mg of the CD38-binding fusion protein is administered to the subject.

In some embodiments, the CD38-binding fusion protein is administered at an amount between 1.5-3 mg/kg of the subject. In some embodiments, the CD38-binding fusion protein is administered at an amount between 120-240 mg to the subject (regardless of the subject's weight). In some embodiments, the CD38-binding fusion protein is administered once every four weeks. In some embodiments, the method further comprises administering to the subject pomalidomide, carfilzomib, daratumumab, lenalidomide, and/or bortezomib.

In some embodiments, the method comprises administering a CD38-binding fusion protein comprising an anti-CD38 antibody comprising a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, and a light chain complementarity determining region 3 (CDR-L3) comprising the amino acid sequence of SEQ ID NO: 6.

In some embodiments, the CD38-binding fusion protein comprises the amino acid sequence of SEQ ID NO: 12.

In some embodiments, the method further comprises administering a corticosteroid. In some embodiments, the corticosteroid is dexamethasone.

In some embodiments, the method further comprises administering an anti-histamine. In some embodiments, the antihistamine is montelukast or diphenhydramine. In some embodiments, the method comprises administering the antihistamine 12 hours before and 1 hour after administering the CD38-binding fusion protein to the subject for a first cycle of treatment and administering the antihistamine 1 hour before and 1 hour after administering the CD38-binding fusion protein to the subject for cycles of treatment after the first cycle of treatment.

In some embodiments, the method further comprises administering an analgesic. In some embodiments, the analgesic is acetaminophen. In some embodiments, the analgesic is an NSAID.

In some aspects, this disclosure describes a method of promoting an immune response in a subject, the method comprising administering the CD38-fusion binding protein, as described herein, to the subject. In some embodiments, promoting the immune response in the subject comprises promoting an immune response at a location of a cancer and/or a tumor e.g., melanoma or multiple myeloma) in the subject.

### BRIEF DESCRIPTION OF DRAWINGS

The drawings are for illustration purposes only, not for limitation.

FIGS. 1A-1E demonstrate the antitumor activity of the CD38-binding fusion protein, including in non-CD38-expressing solid tumors. FIG. 1A shows semi-weekly caliper-based tumor volume measurements in mice bearing CT26 tumors that were treated with vehicle (phosphate-buffered saline) or with exposure matched doses of nontargeted-mATT (10 mg/kg, twice weekly for 2 weeks) or mCD38-mATT (30 mg/kg, twice weekly for 2 weeks). FIGS. 1B and 1C show flow cytometry analysis of CD86 and CD205 expression in CD103+ dendritic cells (DC) harvested from the lymph nodes of mice bearing CT26 tumors 3 days post-treatment with vehicle (phosphate-buffered saline [PBS]), or with exposure matched single doses of nontargeted-mATT (10 mg/kg) or mCD38-mATT (30 mg/kg). FIG. 1D shows flow cytometry analysis of NKp46 expression in CD45+NK cells that were harvested from mice baring CT26 tissue on day 3 post-treatment with vehicle (phosphate-buffered saline [PBS]) or exposure matched single doses of nontargeted-mATT (10 mg/kg) or mCD38-mATT (30 mg/kg). FIG. 1E shows flow cytometry analysis of Ki67 expression in CD8+ T cells harvested from mice baring CT26 tumor on day 6 post-treatment with vehicle (PBS) or exposure matched doses of nontargeted-mATT (10 mg/kg) or mCD38-mATT (30 mg/kg) occurring on Day 0 and Day 3.

FIG. 2 shows a swim plot of overall response over time of the indicated solid tumor types in response to various doses of the CD38-binding fusion protein.

FIGS. 3A-3B shows tumor response in 14 response-evaluable patients with solid tumors treated with the CD38-binding fusion protein. FIG. 3A shows the percent best change from baseline in target lesions as a result of treatment with the indicated dose of the CD38-binding fusion protein in the indicated cancers. FIG. 3B shows the percent change in sum from baseline of target lesion diameters over the indicated time periods as a result of treatment with the indicated dose of the CD38-binding fusion protein.

FIGS. 4A-4B show pharmacokinetics of the CD38-binding fusion protein following administration over two 21-day cycles of treatment in patients with solid tumor.

FIGS. 5A-5C show receptor occupancy and interferon pathway activation in patients at select time points following administration with the indicated doses of the CD38-binding fusion protein in patients with solid tumors. FIG. 5A is flow cytometry analysis showing the dose-dependent saturation of CD38 receptor by the CD38-binding fusion protein at 4 hours post-treatment during day 1 of cycle 1 (C1D1 4 hrs), day 2 of cycle 1 (C1D2), and day 15 of cycle 1 (C1D15) (BL=baseline). FIG. 5B shows induction of the type I interferon (IFN) gene signature at day 2 of cycle 1 (C1D2) and day 1 of cycle 2 (C2D1) calculated as the average of 25 IFN-stimulated genes (FPKM=fragments per kilobase of transcript per million mapped reads; BL=baseline). FIG. 5C shows production of systemic IFNα-mediated cytokine IP-10 at day 1 of cycle 1 (C1D1) and day 2 of cycle 1 (C1D2).

FIGS. 6A-6B show activation of CD8+ T cells and NK cells as a result of CD38-binding fusion protein treatment in patients with solid tumors. FIG. 6A shows analysis of peripheral blood activated and cytolytic CD8+ T cells that were CD69+ and granzyme B+ in patients at day 1 of cycle 1 (C1D1) before first treatment, day 2 of cycle 1 (C1D2), day 8 of cycle 1 (C1D8), day 1 of cycle 2 (C2D1) before second treatment, and day 2 of cycle 2 (C2D2). FIG. 6B shows analysis of peripheral blood activated and cytolytic NK cells that were CD69+ and granzyme B+ in patients at day 1 of cycle 1 (C1D1) before first treatment, day 2 of cycle 1 (C1D2), day 8 of cycle 1 (C1D8), day 1 of cycle 2 (C2D1) before second treatment, and day 2 of cycle 2 (C2D2).

FIG. 7 shows a schematic of a phase 1b/2 study design for testing the safety, tolerability, pharmacokinetics, pharmacodynamics and antitumor activity of the CD38-binding fusion protein as a single agent and in combination with pembrolizumab in adult patients with advanced or metastatic solid tumors (RP2D=recommended phase 2 dose).

FIG. 8A-8C show computational results comparing fixed and body-weight adjusted doses of CD38-binding fusion protein for single-dose maximum observed serum concentration (Cₘₐₓ) and area under the serum curve (AUC) exposure for the overall patient population in the clinical trial. The median weight of patients in the patient population was 80 kg. FIG. 8A shows that a fixed dose of 120 mg is expected to result in similar exposure (AUC) as a 1.5 mg/kg body-weight adjusted dose. FIG. 8B shows that a fixed dose of 240 mg is expected to result in similar Cₘₐₓ as a 3 mg/kg body-weight adjusted dose. FIG. 8C shows that a fixed dose of 240 mg is expected to result in similar exposure (AUC) as a 3 mg/kg body-weight adjusted dose. On the plots, single dots are means. The upper dashed line on FIGS. 8B-8C denotes the exposure based on the noncompartmental analysis of the observed data for a patient of the 3 mg/kg Q4W dose group who exhibited DLT (delayed platelet count recovery). The lower dashed line on FIGS. 8B-8C denotes the mean simulated exposure for 6 mg/kg dose group.

### DETAILED DESCRIPTION

Various terms relating to aspects of disclosure are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

As used herein, “about” means within 5%, e.g., within 5%, 4%, 3%, 2%, or 1% of a given value or range.

As used herein the term “improved”, when referring to improved cancer treatment, improved therapeutic efficacy, etc., may in context refer to an improvement relative to an attribute of the treatment or an attribute of the subject. For example, improved therapeutic efficiency of the CD38-binding fusion protein may be positively correlated with CD38 expression of the tumor cells of the subject. For example, CD38-binding fusion protein therapeutic efficacy may be improved in subjects having higher tumor CD38 expression as compared to subjects having lower tumor CD38 expression. In another example, therapeutic efficacy may improve as a particular dose or dosing regimen of the CD38-binding fusion protein being administrated to a subject achieves greater efficacy and/or reduced side effects compared to a different dose or dosing regimen of the CD38-binding fusion protein. The term “improved” when referring to improved cancer treatment, improved therapeutic efficacy, etc., may in context refer to improvement relative to a control. In some embodiments, the control is a subject's response to a placebo treatment. In some embodiments, the control is a subject's response to standard of care (e.g., standard of care for a cancer and/or tumor of the subject). In some embodiments, the control is an average therapeutic efficacy (e.g., survival time) of the population of patients being administered a given therapeutic (e.g., the CD38-binding fusion protein). In some embodiments, the control is a baseline (e.g., patient status before treatment). In some embodiments, improved therapeutic efficacy is evidenced by an increased survival time of a subject by at least 5% (e.g., at least 10%, at least 20%, at least 30%, at least 40%, at least 50%, at least 60%, at least 70%, at least 80%, at least 90%, at least 100%, at least 150%, at least 200% or more) as compared to a baseline. In some embodiments, improved therapeutic efficacy is evidenced by an increased survival time of a subject by 5-10%, 10-20%, 20-40%, 60-100%, or 100-200% as compared to a baseline. In some embodiments, improved therapeutic efficacy is evidenced by increased rate of disease remission in one or more subjects, as compared to a baseline. In some embodiments, improved therapeutic efficacy is evidenced by augmenting immune cell function in a subject; for example, improving the immune cell's ability to kill cancer cells in the subject.

As used herein the term “promote” when used in the context of immune responses, means stimulate, initiate, augment, or enhance, including releasing from inhibition. In some embodiments, promoting an immune response comprises increasing cytokine and/or chemokine expression in the subject (e.g., increasing cytokine or chemokine expression by at least 10%, at least 20%, at least 30%, at least 40%, at least 50%, at least 60%, at least 70%, at least 80%, at least 90%, at least 100%, at least 150%, at least 200% or more). In some embodiments, promoting an immune response comprises activating interferon alpha signaling. In some embodiments, promoting an immune response comprises inducing an immune response against a cancer and/or tumor of the subject, or inducing an immune response at the location of a cancer and/or a tumor in the subject.

The present disclosure, in some aspects, relates to methods of treating cancer using a CD38-binding fusion protein comprising an anti-CD38 antibody fused to an attenuated interferon alpha-2b protein. In some embodiments, a method described herein comprises administering to a subject in need there of a composition comprising a CD38-binding fusion protein. In some embodiments, the method further comprises administering to the subject an immune checkpoint inhibitor (e.g., a PD-1 inhibitor such as pembrolizumab).

In some embodiments, a method described herein comprises administering to a subject in need thereof a composition comprising a CD38-binding fusion protein, wherein the subject is receiving or has received treatment with an immune checkpoint inhibitor (e.g., a PD-1 inhibitor such as pembrolizumab).

In some embodiments, a method described herein comprises administering to a subject in need thereof a composition comprising an immune checkpoint inhibitor (e.g., a PD-1 inhibitor such as pembrolizumab), wherein the subject is receiving or has received treatment with a CD38-binding fusion protein.

A “CD38-binding fusion protein,” as used herein, refers to a fusion protein comprising a CD38 binding domain fused to one or more (e.g., one, two) attenuated interferon alpha-2b protein. A “fusion protein” refers to a polypeptide comprising two or more proteinaceous components associated by at least one covalent bond which is a peptide bond, regardless of whether the peptide bond involves the participation of a carbon atom of a carboxyl acid group or involves another carbon atom. The term “fuse” refers to the act of creating a fused molecule as described above, such as, e.g., a fusion protein generated from the recombinant fusion of genetic regions which when translated produces a single proteinaceous molecule. CD38-binding fusion proteins that may be used in the compositions described herein are described in the art, e.g., in U.S. patent Ser. No. 10/544,199, incorporated herein by reference. The amino acid sequences for a particular anti-CD38 antibody are provided in Table 1.

A CD38-binding fusion protein used in a method described herein comprises an anti-CD38 antibody. The term “antibody,” as used herein includes, for example, an intact immunoglobulin or an antigen binding portion of an immunoglobulin or an antigen binding protein related or derived from an immunoglobulin. Intact antibody structural units often comprise a tetrameric protein. Each tetramer is typically composed of two identical pairs of polypeptide chains, each pair having one “light” chain (typically having a molecular weight of about 25 kDa) and one “heavy” chain (typically having a molecular weight of about 50- to 70 kDa). Human immunoglobulin light chains may be classified as having kappa or lambda light chains. In some embodiments, the antibodies described herein comprise antigen binding domains (e.g., antibody heavy and/or light chains) that generally are based on the IgG class, which has several subclasses, including, but not limited to IgG1, IgG2, IgG3, and IgG4. In general, IgG1 has different allotypes with polymorphisms at 356 (D or E), IgG2 and 358 (L or M). The sequences depicted herein use the 356D/358M allotype; however any allotype is included herein and can be used in accordance with the present disclosure. For example, any sequence inclusive of an IgG1 Fc domain included herein can have 356E/358L replacing the 356D/358M allotype.

The anti-CD38 antibody of the CD38-binding fusion protein used in a method described herein comprise a heavy chain comprising a heavy chain variable domain (VH) and a light chain comprising a light chain variable domain (VL). A “variable domain,” as used herein, refers to the region of an immunoglobulin that comprises one or more Ig domains substantially encoded by any of the Vκ (V.kappa), Vλ (V.lamda), and/or VH genes that make up the kappa, lambda, and heavy chain immunoglobulin genetic loci, respectively. In the variable domains, three loops are gathered for each of the V domains of the heavy chain and light chain to form an antigen-binding site. Each of the loops is referred to as a complementarity-determining region (hereinafter referred to as a “CDR”). Additionally, the variable domains also contain relatively invariant stretches called framework regions (FRs) of 15-30 amino acids separated by CDRs. Each VH and VL is composed of three CDRs and four FRs, arranged from amino-terminus to carboxy-terminus in the following order: FR1-CDR1-FR2-CDR2-FR3-CDR3-FR4. In some embodiments, an “antibody molecule” refers to two-chain and multi-chain immunoglobulin proteins and glycoproteins. In some embodiments, an anti-CD38 antibody of the CD38-binding fusion protein used in a method described herein is an antibody fragment or antigen binding fragment of an antibody, including, for example, Fab, Fab′, F(ab′)2, and Fv fragments.

In some embodiments, an anti-CD38 antibody of the CD38-binding fusion protein used in a method described herein comprises a VH comprising a CDRH1 comprising the amino acid sequence of SEQ ID NO: 1, a CDRH2 comprising the amino acid sequence of SEQ ID NO: 2, and a CDRH3 comprising the amino acid sequence of SEQ ID NO: 3; and a VL comprising a CDRL1 comprising the amino acid sequence of SEQ ID NO: 4, a CDRL2 comprising the amino acid sequence of SEQ ID NO: 5, and a CDRL3 comprising the amino acid sequence of SEQ ID NO: 6. In some embodiments, an anti-CD38 antibody of the CD38-binding fusion protein used in a method described herein comprises a set of 6 CDRs that collectively contain up to 10 (e.g., 1, 2, 3, 4, 5, 6, 7, 8, 9, or 10) amino acid modifications, relative to the 6 CDRs of the anti-CD38 antibody provided in Table 1. For example, in some embodiments, the CDRs can be modified in any fashion, as long as the total number of changes in the set of 6 CDRs does not exceed 10 amino acid modifications, with any combination of CDRs being changed; e.g., there may be one change in CDRL1, two in CDRH2, none in CDRH3, etc. In some embodiments, each CDR has no more than a single amino acid substitution relative to the corresponding CDR of the anti-CD38 antibody provided in Table 1. In some embodiments, amino acid modifications in the CDRH3 are avoided.

In some embodiments, an anti-CD38 antibody of the CD38-binding fusion protein used in a method described herein comprises a VH comprising the amino acid sequence of SEQ ID NO: 7 and a VL comprising the amino acid sequence of SEQ ID NO: 8. In some embodiments, an anti-CD38 antibody of the CD38-binding fusion protein used in a method described herein comprises a VH comprising an amino acid sequence that is at least 80% (e.g., at least 80%, at least 85%, at least 90%, at least 95%, at least 99%) identical to the amino acid sequence of SEQ ID NO: 7 and a VL comprising an amino acid sequence that is at least 80% (e.g., at least 80%, at least 85%, at least 90%, at least 95%, at least 99%) identical to the amino acid sequence of SEQ ID NO: 8.

In some embodiments, an anti-CD38 antibody of the CD38-binding fusion protein used in a method described herein is a full-length IgG antibody. In a full-length IgG antibody, each heavy chain is comprised of a heavy chain variable region (abbreviated herein as VH) and a heavy chain constant region. Each light chain is comprised of a light chain variable region (abbreviated herein as VL) and a light chain constant region. In some embodiments, the immunoglobulin molecules are IgG class IgG4, or a subclass thereof.

In some embodiments, an anti-CD38 antibody of the CD38-binding fusion protein used in a method described herein comprises an IgG4 constant region (e.g., a human IgG4 constant region comprising the amino acid sequence of SEQ ID NO: 14). As used herein, the term “IgG4 constant region” refers to a wild-type IgG4 constant region (e.g., a wild-type human IgG4 constant region) or an IgG4 constant region variant (e.g., a human IgG4 constant region variant) or fragment thereof. IgG4 constant region variants (e.g., human IgG4 constant region variants) that may be used in the anti-CD38 antibody of the CD38-binding fusion protein used in a method described herein may, in some embodiments, comprise one or more mutations, e.g., mutations that stabilize the hinge region and/or reduce the toxicity of the antibody. For example, a mutation at position 228 of the IgG4 according to the EU numbering system stabilizes the hinge of IgG4. In some embodiments, a mutation at position 228 of the IgG4 constant region according to the EU numbering system results in a proline at position 228.

In some embodiments, mutations in the IgG4 constant region decrease antibody dependent cell cytotoxicity (ADCC). “Antibody dependent cell-mediated cytotoxicity (ADCC),” as used herein, refers to a cell-mediated reaction wherein nonspecific cytotoxic cells that express Fc gamma receptors (FcγRs) recognize bound antibody on a target cell and subsequently cause lysis of the target cell. In some embodiments, an anti-CD38 antibody of the CD38-binding fusion protein used in a method described herein comprises an IgG4 constant region comprising one or more mutations that reduce ADCC to avoid undesirably high levels of cytotoxicity (e.g., mutations at one or more of positions 252, 254, and 256 of the IgG4 constant region according to the EU numbering system). In some embodiments, a mutation at position 252 of the IgG4 constant region according to the EU numbering system results in a tyrosine at position 252. In some embodiments, a mutation at position 254 of the IgG4 constant region according to the EU numbering system results in a threonine at position 254. In some embodiments, a mutation at position 256 of the IgG4 constant region according to the EU numbering system results in a glutamic acid at position 256.

In some embodiments, an anti-CD38 antibody of the CD38-binding fusion protein used in a method described herein comprises an IgG4 constant region comprising a mutation at position 228 of the IgG4 constant region according to the EU numbering system. In some embodiments, an anti-CD38 antibody of the CD38-binding fusion protein used in a method described herein comprises an IgG4 constant region comprising the amino acid sequence of SEQ ID NO: 15.

In some embodiments, an anti-CD38 antibody of the CD38-binding fusion protein used in a method described herein comprises a heavy chain comprising a VH and a human IgG4 constant region, wherein the VH comprises the amino acid sequence of SEQ ID NO: 7 and the IgG4 constant region comprises the amino acid sequence of SEQ ID NO: 15. In some embodiments, an anti-CD38 antibody of the CD38-binding fusion protein used in a method described herein comprises a heavy chain comprising the amino acid sequence of SEQ ID NO: 9. In some embodiments, an anti-CD38 antibody of the CD38-binding fusion protein used in a method described herein comprises a heavy chain comprising an amino acid sequence at least 80% (e.g., at least 80%, at least 85%, at least 90%, at least 95%, at least 99%) identical to the amino acid sequence of SEQ ID NO: 9.

In some embodiments, an anti-CD38 antibody of the CD38-binding fusion protein used in a method described herein comprises a light chain comprising a VL and a kappa light constant region, wherein the VL comprises the amino acid sequence of SEQ ID NO: 8. In some embodiments, an anti-CD38 antibody of the CD38-binding fusion protein used in a method described herein comprises a light chain comprising the amino acid sequence of SEQ ID NO: 10. In some embodiments, an anti-CD38 antibody of the CD38-binding fusion protein used in a method described herein comprises a light chain comprising an amino acid sequence at least 80% (e.g., at least 80%, at least 85%, at least 90%, at least 95%, at least 99%) identical to the amino acid sequence of SEQ ID NO: 10.

In some embodiments, a CD38-binding fusion protein used in a method described herein further comprises an anti-CD38 antibody (e.g., the anti-CD38 antibody provided in Table 1) fused to an attenuated interferon alpha-2b protein (e.g., the attenuated interferon alpha-2b protein is fused to the heavy chain of the anti-CD38 antibody). It has been observed that interferon-alpha-2b can be attenuated in its biologic activity, which is mediated through the interferon binding to an interferon receptor on a cell surface, by introducing certain amino acid changes into the protein sequence. In some embodiments, an attenuated interferon alpha-2b protein comprises mutations that reduce its potency (e.g., A145D) and/or eliminate O-linked glycosylation of the interferon alpha-2b protein (e.g., T106A). An attenuated interferon molecule can be fused to antibodies that specifically bind to CD38 (e.g., an anti-CD38 antibody), as described herein, such that the anti-CD38 antibody may serve as a delivery vehicle for the attenuated interferon to CD38-positive cells with a resulting diminution of off target interferon activity caused by the attenuated interferon molecule.

In some embodiments, the attenuated interferon alpha-2b protein is fused to the heavy chain of the anti-CD38 antibody. In some embodiments, the attenuated interferon alpha-2b protein is fused to the C-terminus of the heavy chain of the anti-CD38 antibody. As such, in some embodiments, the CD38-binding fusion protein used in a method described herein comprises a heavy chain and a light chain, wherein the heavy chain comprises the heavy chain of an anti-CD38 antibody fused to an attenuated interferon alpha-2b protein and wherein the light chain is the light chain of the anti-CD38 antibody. In some embodiments, the CD38-binding fusion protein used in a method described herein comprises two heavy chains and two light chains, wherein each heavy chain comprises the heavy chain of an anti-CD38 antibody fused to an attenuated interferon alpha-2b protein and wherein each light chain is the light chain of the anti-CD38 antibody.

In some embodiments, the attenuated interferon alpha-2b comprises T106A and A145D mutations relative to a wild type human interferon alpha-2b (e.g., a human interferon alpha-2b comprising the amino acid sequence of SEQ ID NO: 11). In some embodiments, the attenuated interferon alpha-2b comprises the amino acid of SEQ ID NO: 12. In some embodiments, the attenuated interferon alpha-2b comprises an amino acid sequence at least 80% (e.g., at least 80%, at least 85%, at least 90%, at least 95%, at least 99%) identical to the amino acid of SEQ ID NO: 12.

In some embodiments, a CD38-binding fusion protein used in a method described herein comprises a heavy chain comprising an amino acid sequence at least 80% (e.g., at least 80%, at least 85%, at least 90%, at least 95%, at least 99%) identical to the amino acid of SEQ ID NO: 13 and a light chain comprising an amino acid sequence at least 80% (e.g., at least 80%, at least 85%, at least 90%, at least 95%, at least 99%) identical to the amino acid of SEQ ID NO: 10. In some embodiments, a CD38-binding fusion protein used in a method described herein comprises the amino acid of SEQ ID NO: 13 and a light chain comprising the amino acid of SEQ ID NO: 10. In some embodiments, a CD38-binding fusion protein used in a method described herein comprises two heavy chains and two light chains, wherein each heavy comprises the amino acid sequence of SEQ ID NO: 13 and each light chain comprises the amino acid sequence of SEQ ID NO: 10.

TABLE 1
CD38-binding fusion protein amino acid sequences

| | | | SEQ ID |
|---|---|---|---|
| | | Sequence | NO |
| anti-CD38 | CDR-H1 | DSVMN | 1 |
| antibody | CDR-H2 | WIDPEYGRTDVAEKFQG | |
| *CDRs | CDR-H3 | TKYNSGYGFPY | 3 |
| are | CDR-L1 | KASQNVDSDVD | 4 |
| according | CDR-L2 | KASNDYT | 5 |
| to Kabat | CDR-L3 | MQSNTHPRT | 6 |
| numbering | VH | EVQLVQSGAEVKKPGATVKISCKVSGYTFTD | 7 |
| | | SVMNWVQQAPGKGLEWMGWIDPEYGRTD | |
| | | VAEKFQGRVTITADTSTDTAYMELSSLRSED | |
| | | TAVYYCARTKYNSGYGFPYWGQGTTVTVSS | |
| | VL | DIQMTQSPSSLSASVGDRVTITCKASQNVDS | 8 |
| | | DVDWYQQKPGKAPKLLIYKASNDYTGVPSR | |
| | | FSGSGSGTDFTFTISSLQPEDIATYYCMQSNT | |
| | | HPRTFGGGTKVEIKR | |
| | HC | EVQLVQSGAEVKKPGATVKISCKVSGYTFTD | 9 |
| | | SVMNWVQQAPGKGLEWMGWIDPEYGRTD | |
| | | VAEKFQGRVTITADTSTDTAYMELSSLRSED | |
| | | TAVYYCARTKYNSGYGFPYWGQGTTVTVSS | |
| | | ASTKGPSVFPLAPCSRSTSESTAALGCLVKD | |
| | | YFPEPVTVSWNSGALTSGVHTFPAVLQSSGL | |
| | | YSLSSVVTVPSSSLGTKTYTCNVDHKPSNTK | |
| | | VDKRVESKYGPPCPPCPAPEFLGGPSVFLFPP | |
| | | KPKDTLMISRTPEVTCVVVDVSQEDPEVQFN | |
| | | WYVDGVEVHNAKTKPREEQFNSTYRVVSVL | |
| | | TVLHQDWLNGKEYKCKVSNKGLPSSIEKTIS | |
| | | KAKGQPREPQVYTLPPSQEEMTKNQVSLTCL | |
| | | VKGFYPSDIAVEWESNGQPENNYKTTPPVLD | |
| | | SDGSFFLYSRLTVDKSRWQEGNVFSCSVMH | |
| | | EALHNHYTQKSLSLSLGK | |
| | LC | DIQMTQSPSSLSASVGDRVTITCKASQNVDS | 10 |
| | | DVDWYQQKPGKAPKLLIYKASNDYTGVPSR | |
| | | FSGSGSGTDFTFTISSLQPEDIATYYCMQSNT | |
| | | HPRTFGGGTKVEIKRTVAAPSVFIFPPSDEQL | |
| | | KSGTASVVCLLNNFYPREAKVQWKVDNAL | |
| | | QSGNSQESVTEQDSKDSTYSLSSTLTLSKAD | |
| | | YEKHKVYACEVTHQGLSSPVTKSFNRGEC | |

| | | |
|---|---|---|
| WT interferon alpha-2b | CDLPQTHSLGSRRTLMLLAQMRRISLFSCLK | 11 |
| | DRHDFGFPQEEFGNQFQKAETIPVLHEMIQQI | |
| | FNLFSTKDSSAAWDETLLDKFYTELYQQLN | |
| | DLEACVIQGVGVTETPLMKEDSILAVRKYFQ | |
| | RITLYLKEKKYSPCAWEVVRAEIMRSFSLST | |
| | NLQESLRSKE | |
| Attenuated interferon alpha- | CDLPQTHSLGSRRTLMLLAQMRRISLFSCLK | 12 |
| 2b (T106A, A145D) | DRHDFGFPQEEFGNQFQKAETIPVLHEMIQQI | |
| | FNLFSTKDSSAAWDETLLDKFYTELYQQLN | |
| | DLEACVIQGVGVAETPLMKEDSILAVRKYFQ | |
| | RITLYLKEKKYSPCAWEVVRDEIMRSFSLST | |
| | NLQESLRSKE | |
| HC of anti-CD38 antibody | EVQLVQSGAEVKKPGATVKISCKVSGYTFTD | 13 |
| fused to Attenuated | SVMNWVQQAPGKGLEWMGWIDPEYGRTD | |
| interferon alpha-2b (T106A, | VAEKFQGRVTITADTSTDTAYMELSSLRSED | |
| A145D) | TAVYYCARTKYNSGYGFPYWGQGTTVTVSS | |
| | ASTKGPSVFPLAPCSRSTSESTAALGCLVKD | |
| | YFPEPVTVSWNSGALTSGVHTFPAVLQSSGL | |
| | YSLSSVVTVPSSSLGTKTYTCNVDHKPSNTK | |
| | VDKRVESKYGPPCPPCPAPEFLGGPSVFLFPP | |
| | KPKDTLMISRTPEVTCVVVDVSQEDPEVQFN | |
| | WYVDGVEVHNAKTKPREEQFNSTYRVVSVL | |
| | TVLHQDWLNGKEYKCKVSNKGLPSSIEKTIS | |
| | KAKGQPREPQVYTLPPSQEEMTKNQVSLTCL | |
| | VKGFYPSDIAVEWESNGQPENNYKTTPPVLD | |
| | SDGSFFLYSRLTVDKSRWQEGNVESCSVMH | |
| | EALHNHYTQKSLSLSLGKCDLPQTHSLGSRRT | |
| | LMLLAQMRRISLFSCLKDRHDFGFPQEEFGNQ | |
| | FQKAETIPVLHEMIQQIFNLFSTKDSSAAWDET | |
| | LLDKFYTELYQQLNDLEACVIQGVGVAETPLM | |
| | KEDSILAVRKYFQRITLYLKEKKYSPCAWEVVR | |
| | DEIMRSFSLSTNLQESLRSKE | |
| Wild type human IgG4 | ASTKGPSVFPLAPCSRSTSESTAALGCLVKD | 14 |
| constant region | YFPEPVTVSWNSGALTSGVHTFPAVLQSSGL | |
| | YSLSSVVTVPSSSLGTKTYTCNVDHKPSNTK | |
| | VDKRVESKYGPPCPSCPAPEFLGGPSVFLFPP | |
| | KPKDTLMISRTPEVTCVVVDVSQEDPEVQFN | |
| | WYVDGVEVHNAKTKPREEQFNSTYRVVSVL | |
| | TVLHQDWLNGKEYKCKVSNKGLPSSIEKTIS | |
| | KAKGQPREPQVYTLPPSQEEMTKNQVSLTCL | |
| | VKGFYPSDIAVEWESNGQPENNYKTTPPVLD | |
| | SDGSFFLYSRLTVDKSRWQEGNVFSCSVMH | |
| | EALHNHYTQKSLSLSLGK | |
| Human IgG4 constant | ASTKGPSVFPLAPCSRSTSESTAALGCLVKD | 15 |
| region variant with S228P | YFPEPVTVSWNSGALTSGVHTFPAVLQSSGL | |
| mutation (EU numbering | YSLSSVVTVPSSSLGTKTYTCNVDHKPSNTK | |
| system) | VDKRVESKYGPPCPPCPAPEFLGGPSVFLFPP | |
| | KPKDTLMISRTPEVTCVVVDVSQEDPEVQFN | |
| | WYVDGVEVHNAKTKPREEQFNSTYRVVSVL | |
| | TVLHQDWLNGKEYKCKVSNKGLPSSIEKTIS | |
| | KAKGQPREPQVYTLPPSQEEMTKNQVSLTCL | |
| | VKGFYPSDIAVEWESNGQPENNYKTTPPVLD | |
| | SDGSFFLYSRLTVDKSRWQEGNVFSCSVMH | |
| | EALHNHYTQKSLSLSLGK | |

In some embodiments, in a method described herein, the CD38-binding fusion protein is administered at 0.1-15 mg/kg (e.g., 0.1-15, 0.5-15, 1-15, 2-15, 3-15, 4-15, 5-15, 6-15, 7-15, 8-15, 9-15, 10-15, 11-15, 12-15, 13-15, 14-15, 0.1-14, 0.5-14, 1-14, 2-14, 3-14, 4-14, 5-14, 6-14, 7-14, 8-14, 9-14, 10-14, 11-14, 12-14, 13-14, 0.1-13, 0.5-13, 1-13, 2-13, 3-13, 4-13, 5-13, 6-13, 7-13, 8-13, 9-13, 10-13, 11-13, 12-13, 0.1-12, 0.5-12, 1-5, 2-12, 3-12, 4-12, 5-12, 6-12, 7-12, 8-12, 9-12, 10-12, 11-12, 0.1-11, 0.5-11, 1-11, 2-11, 3-11, 4-11, 5-11, 6-11, 7-11, 8-11, 9-11, 10-11, 0.1-10, 0.5-10, 1-10, 2-10, 3-10, 4-10, 5-10, 6-10, 7-10, 8-10, 9-10, 0.1-9, 0.5-9, 1-9, 2-9, 2-9, 3-9, 4-9, 5-9, 6-9, 7-9, 8-9, 0.1-8, 0.5-8, 1-8, 2-8, 3-8, 4-8, 5-8, 6-8, 7-8, 0.1-7, 0.5-7, 1-7, 2-7, 3-7, 4-7, 5-7, or 6-7 mg/kg) of the subject. In some embodiments, in a method described herein, the CD38-binding fusion protein is administered at 0, 1, 0.2, 0.4, 0.75, 1, 1.5, 3, or 6, 9, 12, 14, or 15 mg/kg of the subject. In some embodiments, in a method described herein, the CD38 -binding fusion protein is administered at 0.1-6 mg/kg (e.g., 0.1-6, 0.5-6, 1-6, 2-6, 3-6, 4-6, 5-6, 0.1-5, 0.5-5, 1-5, 2-5, 3-5, 4-5, 0.1-4, 0.5-4, 1-4, 2-4, 3-4, 0.1-3, 0.5-3, 1-3, 2-3, 0.1-2, 0.5-2, 1-2, 0.1-1, or 0.5-1 mg/kg) of the subject. In some embodiments, in a method described herein, the CD38-binding fusion protein is administered at 0.1, 0.2, 0.4, 0.75, 1, 1.5, 3, or 6 mg/kg of the subject. In some embodiments, in a method described herein, the CD38-binding fusion protein is administered at 0.1-1.5 mg/kg (e.g., 0.1-1.5, 0.3-1.4, 0.5-1.3, 0.7-1.2, or 0.9-1.1 mg/kg) of the subject. In some embodiments, in a method described herein, the CD38-binding fusion protein is administered at 0.5-2 mg/kg (e.g., 0.5-2, 0.6-1.8, 0.7-1.6, 0.8-1.4, 0.9-1.2 mg/kg) of the subject. In some embodiments, the CD38-binding fusion protein is administered at about 1 mg/kg of the subject. In some embodiments, the CD38-binding fusion protein is administered at about 0.75 mg/kg of the subject.

In some embodiments, the CD38-binding fusion protein is administered at an increasing dose over time (e.g., a dose escalation). In some embodiments, the dose of the CD38-binding fusion protein is increased during a least one cycle of a multiple cycle dosing regimen (e.g., the dose is increased at cycle 3 of 6). In some embodiments, the dose of the CD38-binding fusion protein is increased during consecutive cycles of a dosing regimen e.g., as follows: 0.001 mg/kg (cycle 1), 0.01 mg/kg (cycle 2), 0.1 mg/kg (cycle 3), 0.75 mg/kg (cycle 4), 1.5 mg/kg (cycle 5), 3 mg/kg (cycle 6), 6 mg/kg (cycle 7), 9 mg/kg (cycle 8), and 14 mg/kg (cycle 9). In some embodiments, the dose of the CD38-binding fusion protein is increased over time, e.g., as follows 0.001 mg/kg, 0.01 mg/kg, 0.1 mg/kg, 0.75 mg/kg, 1.5 mg/kg, 3 mg/kg, 6 mg/kg, 9 mg/kg, and 14 mg/kg. In some embodiments, the CD38-binding fusion protein is administered at a dose of 60-120 mg (e.g., 60-120, 60-100, 60-80, 80-120, 80-100, or 100-120 mg) to the subject (e.g., regardless of the subject's weight). In some embodiments, the CD38-binding fusion protein is administered at a dose of 80-120 mg (e.g., 80, 90, 100, 110, or 120 mg) to the subject (e.g., regardless of the subject's weight). In some embodiments, the CD38-binding fusion protein is administered at a dose of about 80 mg to the subject (e.g., regardless of the subject's weight). In some embodiments, the CD38-binding fusion protein is administered at a dose of about 120 mg to the subject (e.g., regardless of the subject's weight). In some embodiments, the CD38-binding fusion protein is administered at a dose of 120-250 mg or 200-260 mg. In some embodiments, the CD38-binding fusion protein is administered at a dose of up to 240 mg (e.g., regardless of the subject's weight). In some embodiments, the CD38-binding fusion protein is administered at a dose of 240 mg.

In some embodiments, in any one of the methods described herein, the CD38-binding fusion protein is administered once every 1-4 weeks (e.g., once a week, once every two weeks, once every three weeks, or once every four weeks). In some embodiments, the CD38-binding fusion protein is administered once per cycle. A cycle may be 1 week (1-week cycle), 2 weeks (2-week cycle), 3 weeks (3-week cycle), or 4 weeks (4-week cycle). In some embodiments, the cycle is a 3-week cycle or a 4-week cycle. In some embodiments, the CD38-binding fusion protein is administered once per cycle for 1, 2, 3, 4, 5, 6, 7, 8, 9, 10, 11, 12, 13, 14, or 15 cycles. In some embodiments, the CD38-binding fusion protein is administered once per cycle for 1-12 cycles. In some embodiments, in any one of the methods described herein, the CD38-binding fusion protein is administered once every three weeks. In some embodiments, in any one of the methods described herein, the CD38-binding fusion protein is administered once every three weeks for a period of time (e.g., up to 1, 2 3, 4, 5 years or more). In some embodiments, in any one of the methods described herein, the CD38-binding fusion protein is administered once every three weeks for the remainder of the patient's lifetime.

In some embodiments, the method comprises monitoring the subject to determine how many cycles of CD38-binding fusion protein to administer (e.g., monitoring treatment efficacy and/or side effects). In some embodiments, the method includes monitoring the subject for side effects (e.g., neutropenia lasting more than 7 consecutive days, thrombocytopenia lasting more than 14 consecutive days, and nausea/vomiting that lasts more than 48 hours) and discontinuing, delaying, or decreasing CD38-binding fusion protein treatment if adverse side effects are observed in the subject. In some embodiments, the method includes monitoring the subject for intolerable side effects and discontinuing, delaying, or decreasing CD38-binding fusion protein treatment if intolerable side effects are observed in the subject.

In some embodiments, increased CD38 expression on the cancer cells and/or the subject's immune cells after administration of the CD38-binding fusion protein is associated with improved cancer treatment efficacy (e.g., improved patient survival). In some embodiments, CD38-expression on the cancer cells is not associated with or weakly associated with CD38-binding fusion protein efficacy. In some embodiments, CD38-binding fusion protein efficacy is independent of CD38 expression on the myeloma cells and immune cells of the bone marrow. In some embodiments, administering the CD38-binding fusion protein increases the number of natural killer cells in the subject blood. In some embodiments, administering the CD38-binding fusion protein activates the subject's immune cells (e.g., T cells).

In some embodiments, increased immune activity in the subject (e.g., neopternin expression, complement expression, cytokine/chemokine expression, and immune-related gene expression) is associated with administration of the CD38-binding fusion protein. In some embodiments, increased immune activity in the subject (e.g., neopternin expression, complement expression, cytokine/chemokine expression, and immune-related gene expression) after administration of the CD38-binding fusion protein is associated with improved cancer treatment efficacy (e.g., patient survival). In some embodiments, administration of the CD38-binding fusion protein increases cytokine/chemokine expression. In some embodiments, administration of the CD38-binding fusion protein increases cytokine/chemokine expression by at least 2-fold (e.g., at least 3-fold, at least 5-fold, or at least 10-fold). In some embodiments, administration of the CD38-binding fusion protein increases expression of one or more of the following genes: IFI44L, RSAD2, IFI27, OAS3, IFIT1, IFI44, EPSTI1, HERC5, OAS1, MX1, OAS2, USP18, SPATS2L, IFIT3, OASL, DDX60, LY6E, IFI6, LAMP3, RTP4, HERC6, SERPING1, CMPK2, CXCL10, and IFIT2, which may be indicative of an interferon response in a subject (Harari et al., Human Molecular Genetics 24.11 (2015): 3192-3205). In some embodiments, administration of the CD38-binding fusion protein increases expression of at least 5 of the following genes: IFI44L, RSAD2, IFI27, OAS3, IFIT1, IFI44, EPSTI1, HERC5, OAS1, MX1, OAS2, USP18, SPATS2L, IFIT3, OASL, DDX60, LY6E, IFI6, LAMP3, RTP4, HERC6, SERPING1, CMPK2, CXCL10, and IFIT2. In some embodiments, administration of the CD38-binding fusion protein increases expression of at least 10 of the following genes: IFI44L, RSAD2, IFI27, OAS3, IFIT1, IFI44, EPSTI1, HERC5, OAS1, MX1, OAS2, USP18, SPATS2L, IFIT3, OASL, DDX60, LY6E, IFI6, LAMP3, RTP4, HERC6, SERPING1, CMPK2, CXCL10, and IFIT2. In some embodiments, administration of the CD38-binding fusion protein increases expression of at least 15 of the following genes: IFI44L, RSAD2, IFI27, OAS3, IFIT1, IFI44, EPSTI1, HERC5, OAS1, MX1, OAS2, USP18, SPATS2L, IFIT3, OASL, DDX60, LY6E, IFI6, LAMP3, RTP4, HERC6, SERPING1, CMPK2, CXCL10, and IFIT2. In some embodiments, administration of the CD38-binding fusion protein increases expression of at least 20 of the following genes: IFI44L, RSAD2, IFI27, OAS3, IFIT1, IFI44, EPSTI1, HERC5, OAS1, MX1, OAS2, USP18, SPATS2L, IFIT3, OASL, DDX60, LY6E, IFI6, LAMP3, RTP4, HERC6, SERPING1, CMPK2, CXCL10, and IFIT2. In some embodiments, administration of the CD38-binding fusion protein increases expression of the following genes: IFI44L, RSAD2, IFI27, OAS3, IFIT1, IFI44, EPSTI1, HERC5, OAS1, MX1, OAS2, USP18, SPATS2L, IFIT3, OASL, DDX60, LY6E, IFI6, LAMP3, RTP4, HERC6, SERPING1, CMPK2, CXCL10, and IFIT2.

In some embodiments, improved immune cell activity in the subject after administration of the CD38-binding fusion protein is associated with improved cancer treatment efficacy (e.g., patient survival). In some embodiments, administering the CD38-binding fusion protein to a subject results in increased anti-CD38-binding fusion protein antibody concentration. In some embodiments, the rate of increase of CD38-binding fusion protein antibody concentration in a subject during administration (e.g., over 1 or more cycles of administration) of the CD38-binding fusion protein is associated with cancer treatment efficacy. For example, a slower rate of increasing CD38-binding fusion protein antibody concentration in a subject may be associated with higher cancer treatment efficacy compared to a higher rate of increasing CD38-binding fusion protein antibody concentration in a subject. In some embodiments, the baseline concentration of CD38-binding fusion protein antibodies in the subject may be indicative of CD38-binding fusion protein cancer treatment efficacy. For example, a lower baseline concentration of CD38-binding fusion protein antibodies in the subject may be associated with higher cancer treatment efficacy compared to a higher baseline concentration of CD38-binding fusion protein antibodies in the subject.

In some embodiments, the subject is monitored for measurable minimal residual disease (MRD) status during administration and/or after administration of a composition comprising the CD38-binding fusion protein. MRD may refer to evidence of cancer (e.g., multiple myeloma or melanoma) persisting or relapsing in a subject (e.g., detection of cancer cells or cancer DNA in the subject) after cancer treatment (e.g., administration of the CD38-binding fusion protein or tumor resection). MRD may be detected using any suitable method, including but not limited to, quantitative polymerase chain reaction (qPCR), flow cytometry, or next-generation sequencing. Negative MRD status may indicate that MRD is not present or not detectable above a threshold in a subject. For example, negative MRD status of a subject may be determined when MRD is not detected at a sensitivity of detection of 10⁻⁵ or 10⁻⁶. In some embodiments, negative MRD status is determined when MRD is not detected at a sensitivity of 10⁻⁵ in a subject that achieved a complete response (e.g., the disappearance of all signs of cancer in response to treatment) to a CD38-binding fusion protein treatment. In some embodiments, a rate to negative MRD status is measured during the cycles of administration of the composition comprising CD38-binding fusion protein. In some embodiments, the duration of negative MRD status is monitored in a subject who had a complete response to a composition comprising the CD38-binding fusion protein. In some embodiments, the duration of negative MRD status is greater when a subject is administered a composition comprising a CD38-binding fusion protein and is also receiving treatment with an anti-PD-1 antibody as compared to that for a subject administered a composition comprising the CD38-binding fusion protein but not receiving an anti-PD-1 antibody. In some embodiments, the duration of negative MRD status is at least 1, at least 2, at least 3, at least 4, at least 5, at least 6, at least 7, at least 8, at least 9, at least 10 or more years.

In some embodiments, in any one of the methods described herein, a dose of the CD38-binding fusion protein is administered (e.g., intravenously administered) over 0.5 hours, 1 hour, 2 hours, 3 hours, 4 hours, 5 hours, 6 hours, or longer. In some embodiments, a dose of the CD38-binding fusion protein is administered over at least 0.5 hours, at least 1 hour, at least 2 hours, at least 3 hours, at least 4 hours, at least 5 hours, at least 6 hours. In some embodiments, a dose of the CD38-binding fusion protein is administered over 0.5-7 hours, 1-7 hour, 2-7 hours, 3-7 hours, 4-7 hours, 5-7 hours, 6-7 hours, or longer. In some embodiments, a dose of the CD38-binding fusion protein is administered over 0.5-4 hours. In some embodiments, a dose of the CD38-binding fusion protein is administered over no more than 7 hours. In some embodiments, a dose of the CD38-binding fusion protein that is less than or equal to 6 mg/kg is administered over 1 hour. In some embodiments, a dose of the CD38-binding fusion protein that is less than 6 mg/kg (of the subject) is administered over 1 hour. In some embodiments, a dose of the CD38-binding fusion protein that is greater or equal to than 6 mg/kg (of the subject) is administered over 2 hours. In some embodiments, a dose of the CD38-binding fusion protein that is greater than 6 mg/kg (of the subject) is administered for 2 hours.

In some embodiments, in any one of the methods described herein, comprises administering the CD38-binding fusion protein to a subject that is refractory to or relapsed after standard cancer treatments (e.g., standard multiple myeloma treatments). In some embodiments, the subject is refractory to one or more of anti-CD38 antibodies (e.g., Daratumumab or Isatuximab), CAR-T cell therapies, anti-BCMA therapies, Immunomodulator (IMiD) therapies, proteasome inhibitor therapies, or Elotuzumab. In some embodiments, the subject is refractory to the last line of prior therapy.

In some embodiments, any one of the methods described herein further comprises administering to the subject a corticosteroid. In some embodiments, the corticosteroid is cortisone, prednisone, methylprednisone, prednisolone, methylprednisolone, dexamethasone, betamethasone or hydrocortisone. In some embodiments, the corticosteroid is dexamethasone. In some embodiments, the method comprises administering 10-50 mg, 10-40 mg, 10-30 mg, 10-20 mg, 20-50 mg, 30-50 mg, or 35-45 mg of dexamethasone to the subject. In some embodiments, the method comprises administering 10, 15, 20, 25, 30, 35, 40, 45, or 50 mg of the dexamethasone to the subject. In some embodiments, the method comprises administering 40 mg of the dexamethasone to a subject that is younger than 75 years old. In some embodiments, the method comprises administering 20 mg of dexamethasone to a subject that is 75 years-old or older. In some embodiments, the method comprises administering 20 mg of dexamethasone to the subject. In some embodiments, the corticosteroid is methylprednisone. In some embodiments, the method comprises administering 50-150 mg, 50-140 mg, 50-130 mg, 50-120 mg, 75-150 mg, 75-125 mg, or 90-110 mg of methylprednisone to the subject. In some embodiments, the method comprises administering 50, 75, 90, 95 100, 105, 115, 125, or 150 mg of the methylprednisone to the subject. In some embodiments, the method comprises administering 100 mg of the methylprednisone to the subject. In some embodiments, when the subject does not experience significant infusion-related reaction (IRR), the dose of the corticosteroid may be reduced in half (e.g., reduced from 100 mg of methylprednisone to 50 mg of methylprednisone; or reduced from 20 mg of dexamethasone to 10 mg of dexamethasone). In some embodiments, when the subject does not experience significant infusion-related reaction (IRR) after 3 administrations (i.e., 3 doses) of the CD38-binding fusion protein, the dose of the steroid may be reduced in half for the remaining administrations (e.g., administration 4 to the end of administration).

In some embodiments, the method comprises administering the corticosteroid (e.g., dexamethasone or methylprednisone) twice a week. In some embodiments, the method comprises administering the corticosteroid (e.g., dexamethasone or methylprednisone)) once a week. In some embodiments, the method comprises administering the corticosteroid (e.g., dexamethasone or methylprednisone)) once every two weeks. In some embodiments, the method comprises administering the corticosteroid (e.g., dexamethasone or methylprednisone)) once every three weeks. In some embodiments, the method comprises administering the corticosteroid (e.g., dexamethasone or methylprednisone)) with the CD38-binding fusion protein. In some embodiments, the method comprises administering the corticosteroid (e.g., dexamethasone or methylprednisone) to the subject before administration of the CD38-binding fusion protein to the subject. In some embodiments, the method comprises administering the corticosteroid (e.g., dexamethasone or methylprednisone) 1-2 hours before the CD38-binding fusion protein is administered. In some embodiments, the method comprises administering the corticosteroid (e.g., dexamethasone or methylprednisone) to combat inflammation, as needed (e.g., as determined by a medical doctor).

In some embodiments, the method further comprises administering an antihistamine. In some embodiments, the antihistamine is montelukast or diphenhydramine. In some embodiments, 10-70, 20-60, 25-50, or 20-30, or 45-55 mg of diphenhydramine is administered to the subject (or an equivalent amount of montelukast). In some embodiments, 25-50 mg of diphenhydramine is administered to the subject (or an equivalent amount of montelukast). In some embodiments, 20, 25, 30, 35, 40, 45, 45, 50, or 55 mg of diphenhydramine is administered to the subject (or an equivalent amount of montelukast). In some embodiments, 10 mg of montelukast is administered to the subject (e.g., when the subject is intolerant to diphenhydramine, or diphenhydramine is not effective for the subject. In some embodiments, the method comprises administering the antihistamine to the subject before and after administration of the CD38-binding fusion protein to the subject. In some embodiments, the antihistamine is administered 10-14 hours before and 0.5-3 hours after administering the CD38-binding fusion protein to the subject. In some embodiments, the antihistamine is administered 12 hours before and 1 hour after administering the CD38-binding fusion protein to the subject. In some embodiments, the antihistamine is administered 12 hours before and 1 hour after administering the CD38-binding fusion protein to the subject for the first cycle of treatment, and the antihistamine is administered 1 hour before and 1 hour after administering the CD38-binding fusion protein to the subject for the cycles after the first cycle (e.g., cycles 2-12). In some embodiments, the antihistamine is administered 12 hours before and 1 hour after administering the CD38-binding fusion protein to the subject for the first cycle of treatment, and the antihistamine is administered 1 hour before administering the CD38-binding fusion protein to the subject for the cycles after the first cycle (e.g., cycles 2-12).

In some embodiments, the method further comprises administering (or co-administering) an analgesic. In some embodiments, the method comprises administering the analgesic acetaminophen. In some embodiments, 500-1200 mg, 600-1200 mg, 700-1200 mg, 800-1200 mg, 900-1200 mg, 500-1100 mg, 500-1000 mg, 500-900 mg, 500-800 mg, 500-700 mg, 500-600 mg of acetaminophen is administered to the subject. In some embodiments, 650-1000 mg of acetaminophen is administered to the subject. In some embodiments, 500, 600, 700, 800, 900, 1000, 1100, or 1200 mg of acetaminophen is administered to the subject. In some embodiments, the acetaminophen is administered to the subject 1-2 hours before the CD38-binding fusion protein administered. In some embodiments, 650-1000 mg of acetaminophen is administered to the subject 1-2 hours before the CD38-binding fusion protein is administered to the subject.

In some embodiments, the method further comprises administering a non-steroidal anti-inflammatory drug (NSAID) analgesic to the subject. In some embodiments, the NSAID is aspirin, ibuprofen, naproxen, or celecoxib. In some embodiments, 350-650 mg of aspirin is administered to the subject. In some embodiments, 200-600 mg of ibuprofen is administered to the subject. In some embodiments, 200-600 mg of ibuprofen is administered to the subject. In some embodiments, 275-550 mg of naproxen is administered to the subject. In some embodiments, 100-400 mg of celecoxib is administered to the subject.

In some embodiments, any one of the methods described herein further comprises administering to the subject an immune checkpoint inhibitor. As used herein, the term “immune checkpoint inhibitor” refers to molecules that totally or partially reduce, inhibit, interfere with or modulate one or more checkpoint proteins. The immune system has multiple inhibitory pathways that function to maintain self-tolerance and modulate immune responses. For T-cells, the amplitude and quality of response are initiated through antigen recognition by the T-cell receptor and are regulated by immune checkpoint proteins that balance co-stimulatory and inhibitory signals. Checkpoint proteins regulate T-cell activation or function. Numerous checkpoint proteins are known, such as CTLA-4 and its ligands CD80 and CD86; and PD-1 with its ligands PDL1 and PDL2 (Pardoll, Nature Reviews Cancer 12: 252-264, 2012); lymphocyte activation gene-3 (LAG-3), such as B7 (e.g., B7-H3 and B7-H4), and TIM3. These proteins are responsible for co-stimulatory or inhibitory interactions of T-cell responses. Immune checkpoint proteins regulate and maintain self-tolerance and the duration and amplitude of physiological immune responses. Immune checkpoint inhibitors include, but are not limited to, antibodies or molecules that are derived from antibodies.

In some embodiments, the immune checkpoint inhibitor used in any one of the methods described comprises an inhibitor for programmed cell death 1 (PD-1). PD-1 limits the activity of T cells in peripheral tissues at the time of an inflammatory response to infection and to limit autoimmunity PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 12: 252-264, 2012). Examples of PD-1 blockers are described in U.S. Pat. Nos. 7,488,802; 8,008,449; 8,168,757, and PCT Published Patent Application Nos: WO 2008/156712, WO 2010/089411, WO 2010/036959, WO 2011/159877, and WO 2011/082400.

In some embodiments, the PD-1 inhibitor used in any one of the methods described herein is an anti-PD-1 antibody. Non-limiting examples of anti-PD-1 antibodies that may be used in accordance with the present disclosure include: pembrolizumab, nivolumab, pidilizumab, and cemiplimab.

In some embodiments, any one of the methods described herein further comprises administering to the subject an immune checkpoint inhibitor (e.g., a PD-1 inhibitor such as pembrolizumab) together with the CD38-binding fusion protein (e.g., in the same composition) or as a separate administration (e.g., before or after the administration of the CD38-binding fusion protein). In some embodiments, the immune checkpoint inhibitor is administered according to its own administration schedule that is separate from the schedule for administering the CD38-binding fusion protein.

In some embodiments, in any one of the methods described herein, the immune checkpoint inhibitor (e.g., a PD-1 inhibitor such as pembrolizumab) is administered at a dose of 400 mg to the subject (e.g., regardless of the subject's weight) once every six weeks. In some embodiments, in any one of the methods described herein, the immune checkpoint inhibitor (e.g., a PD-1 inhibitor such as pembrolizumab) is administered at a dose of 200 mg to the subject (e.g., regardless of the subject's weight) once every three weeks.

In some embodiments, a method described herein comprises administering to a subject in need thereof a composition comprising a CD38-binding fusion protein at a dose of 0.1-1.5 mg/kg (e.g., 0.75 mg/kg or 1 mg/kg) of the subject once every three weeks, and further administering to a subject an immune checkpoint inhibitor (e.g., a PD-1 inhibitor such as pembrolizumab) at a dose of 400 mg once every six weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof (e.g., a subject having melanoma) a composition comprising a CD38-binding fusion protein at a dose of 0.1-1.5 mg/kg (e.g., 0.75 mg/kg or 1 mg/kg) of the subject once every three weeks, and further administering to a subject an immune checkpoint inhibitor (e.g., a PD-1 inhibitor such as pembrolizumab) at a dose of 200 mg (e.g., regardless of the subject's weight) once every three weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof a composition comprising 80 mg of CD38-binding fusion protein (e.g., regardless of the subject's weight) once every three weeks, and further administering to a subject an immune checkpoint inhibitor (e.g., a PD-1 inhibitor such as pembrolizumab) at a dose of 400 mg (e.g., regardless of the subject's weight) once every six weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof a composition comprising 120 mg of CD38-binding fusion protein (e.g., regardless of the subject's weight) once every three weeks, and further administering to a subject an immune checkpoint inhibitor (e.g., a PD-1 inhibitor such as pembrolizumab) at a dose of 200 mg (e.g., regardless of the subject's weight) once every three weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof a composition comprising a CD38-binding fusion protein at a dose of 0.1-1.5 mg/kg (e.g., 0.75 mg/kg or 1 mg/kg) of the subject once every three weeks, wherein the subject is receiving or has received treatment with an immune checkpoint inhibitor (e.g., a PD-1 inhibitor such as pembrolizumab) at a dose of 400 mg once every six weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof a composition comprising a CD38-binding fusion protein at a dose of 0.1-1.5 mg/kg (e.g., 0.75 mg/kg or 1 mg/kg) of the subject once every three weeks, wherein the subject is receiving or has received treatment with an immune checkpoint inhibitor (e.g., a PD-1 inhibitor such as pembrolizumab) at a dose of 200 mg (e.g., regardless of the subject's weight) once every three weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof a composition comprising 80 mg of CD38-binding fusion protein (e.g., regardless of the subject's weight) once every three weeks, wherein the subject is receiving or has received treatment with an immune checkpoint inhibitor (e.g., a PD-1 inhibitor such as pembrolizumab) at a dose of 400 mg (e.g., regardless of the subject's weight) once every six weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof a composition comprising 120 mg of CD38-binding fusion protein (e.g., regardless of the subject's weight) once every three weeks, wherein the subject is receiving or has received treatment with an immune checkpoint inhibitor (e.g., a PD-1 inhibitor such as pembrolizumab) at a dose of 200 mg (e.g., regardless of the subject's weight) once every three weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof an immune checkpoint inhibitor (e.g., a PD-1 inhibitor such as pembrolizumab) at a dose of 400 mg once every six weeks, wherein the subject is receiving or has received treatment with a composition comprising a CD38-binding fusion protein at a dose of 0.1-1.5 mg/kg (e.g., 0.75 mg/kg or 1 mg/kg) of the subject once every three weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof an immune checkpoint inhibitor (e.g., a PD-1 inhibitor such as pembrolizumab) at a dose of 200 mg (e.g., regardless of the subject's weight) once every three weeks, wherein the subject is receiving or has received treatment with a composition comprising a CD38-binding fusion protein at a dose of 0.1-1.5 mg/kg (e.g., 0.75 mg/kg or 1 mg/kg) of the subject once every three weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof an immune checkpoint inhibitor (e.g., a PD-1 inhibitor such as pembrolizumab) at a dose of 400 mg (e.g., regardless of the subject's weight) once every six weeks, wherein the subject is receiving or has received treatment with a composition comprising 80 mg of CD38 -binding fusion protein (e.g., regardless of the subject's weight) once every three weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or a NSAID).

In some embodiments, a method described herein comprises administering to a subject in need thereof an immune checkpoint inhibitor (e.g., a PD-1 inhibitor such as pembrolizumab) at a dose of 200 mg (e.g., regardless of the subject's weight) once every three weeks, wherein the subject is receiving or has received treatment with a composition comprising 120 mg of CD38-binding fusion protein (e.g., regardless of the subject's weight) once every three weeks. In some embodiments, the administration period is up to 2 years. In some embodiments, the method further comprises administering a corticosteroid (e.g., dexamethasone) and an antihistamine (e.g., diphenhydramine) as described herein. In some embodiments, the method further comprises administering at least one analgesic (e.g., acetaminophen or an NSAID).

In some embodiments, the method comprises administering to a subject having a solid tumor (e.g., melanoma) (1) CD38-binding fusion protein at 0.2-1.5 mg/kg of the subject; (2) pembrolizumab; (3) methylprednisone or dexamethasone; (4) acetaminophen or NSAID; and (5) diphenhydramine or montelukast.

In some embodiments, the method comprises administering to a subject having a solid tumor (e.g., melanoma) (1) CD38-binding fusion protein at 0.2-1.5 mg/kg of the subject; (2) 100-500 mg pembrolizumab; (3) 50-100 mg of methylprednisone or 10-20 mg of dexamethasone; (4) 650-1000 mg of acetaminophen; and (5) 25-50 mg diphenhydramine (or an equivalent dose of montelukast). In some embodiments, the CD38-binding fusion protein is administered to the subject having the solid tumor (e.g., melanoma) once every 3 weeks. In some embodiments, the pembrolizumab is administered to the subject having the solid tumor (e.g., melanoma) once every 6 weeks.

In some embodiments, the method comprises administering to a subject having a solid tumor (e.g., melanoma) (1) CD38-binding fusion protein at 0.2-1.5 mg/kg of the subject; (2) 400 mg of pembrolizumab; (3) 50-100 mg of methylprednisone, or 10-20 mg of dexamethasone; (4) 650-1000 mg of acetaminophen; and (5) 25-50 mg diphenhydramine (or an equivalent dose of montelukast).

In some embodiments, the method comprises administering to a subject having a solid tumor (e.g., melanoma) (1) CD38-binding fusion protein at 1 mg/kg of the subject; (2) 400 mg of pembrolizumab; (3) 50-100 mg of methylprednisone, or 10-20 mg of dexamethasone; (4) 650-1000 mg of acetaminophen; and (5) 25-50 mg diphenhydramine (or an equivalent dose of montelukast).

In some embodiments, the method comprises administering to a subject having a solid tumor (e.g., melanoma) (1) CD38-binding fusion protein at 1 mg/kg of the subject; (2) 400 mg of pembrolizumab; (3) 50-100 mg of methylprednisone; (4) 650-1000 mg of acetaminophen; and (5) 25-50 mg diphenhydramine.

In some embodiments, the method comprises administering to a subject having a solid tumor (e.g., melanoma) (1) CD38-binding fusion protein at 1 mg/kg of the subject; (2) 400 mg of pembrolizumab; (3) 10-20 mg of dexamethasone; (4) 650-1000 mg of acetaminophen; and (5) 25-50 mg diphenhydramine (or an equivalent dose of montelukast).

In some embodiments, the method comprises administering to a subject having a solid tumor (e.g., melanoma): (1) CD38-binding fusion protein at 1 mg/kg of the subject, wherein the CD38-binding fusion protein is administered once every 3 weeks (e.g., administered on day 1 of a 3-week cycle); (2) 400 mg of pembrolizumab, wherein the pembrolizumab is administered once every 6 weeks; (3) 10-20 mg of dexamethasone or 50-100 mg of methylprednisone, wherein the dexamethasone or the methylprednisone is administered 1-2 hours before the CD38-binding fusion protein is administered; (4) 650-1000 mg of acetaminophen, wherein the acetaminophen and is administered 1-2 hours before the CD38-binding fusion protein is administered; and (5) 25-50 mg diphenhydramine (or an equivalent dose of montelukast), wherein the diphenhydramine (or an equivalent dose of montelukast) is administered 12 hours before and 1 hour after administering the CD38-binding fusion protein to the subject for the first cycle of treatment, and the diphenhydramine (or an equivalent dose of montelukast) is administered 1 hour before administering the CD38-binding fusion protein to the subject for the cycles after the first cycle (e.g., cycles 2-12). In some embodiments, the CD38-binding fusion protein comprises and anti-CD38 antibody comprising: a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, and a light chain complementarity determining region 3 (CDR-L3) comprising the amino acid sequence of SEQ ID NO: 6. In some embodiments, the method comprises treating a subject that has cancer (e.g., cutaneous melanoma).

In some embodiments, the method comprises administering to a subject having multiple myeloma (1) CD38-binding fusion protein at 1.5-3 mg/kg of the subject; (2) methylprednisone, dexamethasone; (3) acetaminophen or an NSAID; and (4) diphenhydramine (or an equivalent dose of montelukast).

In some embodiments, the method comprises administering to a subject having multiple myeloma a CD38-binding fusion protein at 1.5-3 mg/kg of the subject or administering a dose of 120-240 mg of the CD38-binding fusion protein to the subject. In some embodiments, the method comprises administering to the subject having multiple myeloma a CD38-binding fusion protein at 1.5, 1.75, 2, 2.25, 2.5, 2.75 or 3 mg/kg of the subject. In some embodiments, the method comprises administering a dose of 120, 160, 190, 210, or 240 mg of CD38-binding fusion protein to a subject having multiple myeloma. In some embodiments, administration of the CD38-binding fusion protein to the subject having multiple myeloma is performed every 4 weeks.

In some embodiments, the method comprises administering to a subject having multiple myeloma (1) a CD38-binding fusion protein at 1.5-3 mg/kg of the subject; (2) 50-100 mg of methylprednisone or 10-20 mg of dexamethasone; (3) 650-1000 mg of acetaminophen; and (4) 25-50 mg diphenhydramine (or an equivalent dose of montelukast).

In some embodiments, the method comprises administering to a subject having multiple myeloma (1) a CD38-binding fusion protein at an amount between 1.5-3 mg/kg of the subject; (2) 50-100 mg of methylprednisone or 10-20 mg of dexamethasone; (3) 650-1000 mg of acetaminophen; and (4) 25-50 mg diphenhydramine (or an equivalent dose of montelukast).

In some embodiments, the method comprises administering to a subject having multiple myeloma (1) a CD38-binding fusion protein at an amount between 1.5, 1.75, 2, 2.25, 2.5, 2.75 or 3 mg/kg of the subject; (2) 50-100 mg of methylprednisone or 10-20 mg of dexamethasone; (3) 650-1000 mg of acetaminophen; and (4) 25-50 mg diphenhydramine (or an equivalent dose of montelukast).

In some embodiments, the method comprises administering to a subject having multiple myeloma (1) a CD38-binding fusion protein at 1.5 mg/kg of the subject; (2) 50-100 mg of methylprednisone or 10-20 mg of dexamethasone; (3) 650-1000 mg of acetaminophen; and (4) 25-50 mg diphenhydramine (or an equivalent dose of montelukast). In some embodiments, administering the CD38-binding fusion protein at at least 1.5 mg/kg of the subject having multiple myeloma achieves therapeutic efficacy. In some embodiments, administering the CD38-binding fusion protein at at least 1.0 mg/kg of the subject having multiple myeloma achieves therapeutic efficacy. In some embodiments, administering the CD38-binding fusion protein at more than 0.75 mg/kg of the subject having multiple myeloma achieves therapeutic efficacy.

In some embodiments, the method comprises administering to a subject having multiple myeloma (1) CD38-binding fusion protein at 1.5, 1.75, 2, 2.25, 2.5, 2.75 or 3 mg/kg of the subject; (2) 50-100 mg of methylprednisone, or 10-20 mg of dexamethasone; (3) 650-1000 mg of acetaminophen; and (4) 25-50 mg diphenhydramine (or an equivalent dose of montelukast).

In some embodiments, the method comprises administering to a subject having multiple myeloma: (1) a CD38-binding fusion protein at 1.5, 1.75, 2, 2.25, 2.5, 2.75 or 3 mg/kg of the subject, wherein the CD38-binding fusion protein is administered once every 4 weeks (e.g., administered on day 1 of a 4-week cycle); (2) 10-20 mg of dexamethasone or 50-100 mg of methylprednisone, wherein the dexamethasone or the methylprednisone is administered 1-2 hours before the CD38-binding fusion protein is administered; (3) 650-1000 mg of acetaminophen, wherein the acetaminophen and is administered 1-2 hours before the CD38-binding fusion protein is administered; and (4) 25-50 mg diphenhydramine (or an equivalent dose of montelukast), wherein the diphenhydramine (or an equivalent dose of montelukast) is administered 12 hours before and 1 hour after administering the CD38-binding fusion protein to the subject for the first cycle of treatment, and the diphenhydramine (or an equivalent dose of montelukast) is administered 1 hour before administering the CD38-binding fusion protein to the subject for the cycles after the first cycle (e.g., cycles 2-12). In some embodiments, the CD38-binding fusion protein comprises and anti-CD38 antibody comprising: a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, and a light chain complementarity determining region 3 (CDR-L3) comprising the amino acid sequence of SEQ ID NO: 6.

In some embodiments, the method comprises administering to the subject having multiple myeloma 120-240 mg of CD38-binding fusion protein. In some embodiments, the method comprises administering to the subject having multiple myeloma 60, 80, 120, or 240 mg of CD38-binding fusion protein. In some embodiments, the method comprises administering to the subject having multiple myeloma 120 mg of CD38-binding fusion protein. In some embodiments, the method comprises administering to the subject having multiple myeloma 240 mg of CD38-binding fusion protein. In some embodiments, the method comprises administering to the subject having multiple myeloma (1) 120-240 mg of CD38-binding fusion protein; (2) 50-100 mg of methylprednisone, or 10-20 mg of dexamethasone; (3) 650-1000 mg of acetaminophen; and (4) 25-50 mg diphenhydramine (or an equivalent dose of montelukast). In some embodiments, administration to the subject having multiple myeloma is performed every 4 weeks.

In some embodiments, the method comprises administering to a subject having multiple myeloma 120, 130, 140, 150, 160, 170, 180, 190, 200, 220, or 240 mg of CD38-binding fusion protein. In some embodiments, the method comprises administering to a subject having multiple myeloma 60, 80, 120, or 240 mg of CD38-binding fusion protein. In some embodiments, the method comprises administering to a subject having multiple myeloma 120 mg of CD38-binding fusion protein. In some embodiments, the method comprises administering to a subject having multiple myeloma 240 mg of CD38-binding fusion protein. In some embodiments, the method comprising administering to a subject having multiple myeloma a dose of the CD38-binding fusion protein once every 4 weeks.

In some embodiments, the method comprises administering to a subject having multiple myeloma (1) 120, 130, 140, 150, 160, 170, 180, 190, 200, 220, or 240 mg of CD38-binding fusion protein; (2) 50-100 mg of methylprednisone, or 10-20 mg of dexamethasone; (3) 650-1000 mg of acetaminophen; and (4) 25-50 mg diphenhydramine (or an equivalent dose of montelukast).

In some embodiments, the method comprises administering to a subject having multiple myeloma (1) a CD38-binding fusion protein at 120 mg; (2) 50-100 mg of methylprednisone, or 10-20 mg of dexamethasone; (3) 650-1000 mg of acetaminophen; and (4) 25-50 mg diphenhydramine (or an equivalent dose of montelukast).

In some embodiments, the method comprises administering to a subject having multiple myeloma (1) a CD38-binding fusion protein at 240 mg; (2) 50-100 mg of methylprednisone, or 10-20 mg of dexamethasone; (3) 650-1000 mg of acetaminophen; and (4) 25-50 mg diphenhydramine (or an equivalent dose of montelukast).

In some embodiments, the method comprises administering to a subject having multiple myeloma (1) a CD38-binding fusion protein at 120, 130, 140, 150, 160, 170, 180, 190, 200, 220, or 240 mg; (2) 50-100 mg of methylprednisone, or 10-20 mg of dexamethasone; (3) 650-1000 mg of acetaminophen; and (4) 25-50 mg diphenhydramine (or an equivalent dose of montelukast).

In some embodiments, the method comprises administering to a subject having multiple myeloma: (1) a CD38-binding fusion protein at 120, 130, 140, 150, 160, 170, 180, 190, 200, 220, and 240 mg, wherein the CD38-binding fusion protein is administered once every 4 weeks (e.g., administered on day 1 of a 4-week cycle); (2) 10-20 mg of dexamethasone or 50-100 mg of methylprednisone, wherein the dexamethasone or the methylprednisone is administered 1-2 hours before the CD38-binding fusion protein is administered; (3) 650-1000 mg of acetaminophen, wherein the acetaminophen and is administered 1-2 hours before the CD38-binding fusion protein is administered; and (4) 25-50 mg diphenhydramine (or an equivalent dose of montelukast), wherein the diphenhydramine (or an equivalent dose of montelukast) is administered 12 hours before and 1 hour after administering the CD38-binding fusion protein to the subject for the first cycle of treatment, and the diphenhydramine (or an equivalent dose of montelukast) is administered 1 hour before administering the CD38-binding fusion protein to the subject for the cycles after the first cycle (e.g., cycles 2-12). In some embodiments, the CD38-binding fusion protein comprises an anti-CD38 antibody comprising: a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, and a light chain complementarity determining region 3 (CDR-L3) comprising the amino acid sequence of SEQ ID NO: 6.

In some embodiments, the method comprises administering to a subject having multiple myeloma a CD38-binding fusion protein at 1.5-6.0 mg/kg of the subject. In some embodiments, the method comprises administering to the subject having multiple myeloma a CD38-binding fusion protein at 3.0-6.0 mg/kg of the subject. In some embodiments, the method comprises administering to a subject having multiple myeloma a CD38-binding fusion protein at a concentration between 3.0-6.0 mg/kg of the subject. In some embodiments, the method comprises administering to a subject having multiple myeloma a CD38-binding fusion protein at 6.0 mg/kg of the subject. In some embodiments, the method comprises administering to a subject having multiple myeloma a CD38-binding fusion protein at 3.0 mg/kg, 3.5 mg/kg, 4.0 mg/kg, 4.5 mg/kg, 5.0 mg/kg, or 5.5 mg/kg of the subject. In some embodiments, administration to the subject having multiple myeloma is performed every 4 weeks.

In some embodiments, the method comprises administering to a subject having multiple myeloma a CD38-binding fusion protein at 3.0 mg/kg, 3.5 mg/kg, 4.0 mg/kg, 4.5 mg/kg, 5.0 mg/kg, or 5.5 mg/kg, of the subject, wherein the CD38-binding fusion protein is administered once every 4 weeks (e.g., administered on day 1 of a 4-week cycle), and the CD38-binding fusion protein comprises an anti-CD38 antibody comprising: a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, and a light chain complementarity determining region 3 (CDR-L3) comprising the amino acid sequence of SEQ ID NO: 6. In some embodiments, the method comprises further administering a corticosteroid (e.g., dexamethasone) to the subject. In some embodiments, the method further comprises administering pomalidomide, carfilzomib, daratumumab, lenalidomide, or bortezomib to the subject having multiple myeloma. In some embodiments, when carfilzomib, daratumumab, lenalidomide, or bortezomib are administered to a subject having multiple myeloma, 40-100 mg of the CD38-binding fusion protein is administered to the subject. In some embodiments, when carfilzomib, daratumumab, lenalidomide, or bortezomib are administered to a subject having multiple myeloma, 60 mg or 80 mg, 120 mg, or 240 mg of the CD38-binding fusion protein is administered to the subject.

In some embodiments, subjects having multiple myeloma can tolerate a higher dose of the CD38-binding fusion protein as compared to subjects having melanoma. Subjects having multiple myeloma may have fewer adverse side effects to higher doses of the CD38-binding fusion protein as compared to subjects having melanoma. In some embodiments, the CD38-binding fusion protein has greater therapeutic efficacy at lower doses in subjects having melanoma compared to subjects having multiple myeloma.

In some embodiments, a composition comprising the CD38-binding fusion protein used in a method described herein comprises a buffer (e.g., a histidine/histidine-HCl buffer), a tonicity agent (e.g., arginine-HCl), a stabilizer (e.g., sucrose), and a surfactant (e.g., polysorbate such as polysorbate 80). A buffer may have stabilizing properties. A tonicity agent may have stabilizing properties. A surfactant may have stabilizing properties.

In some embodiments, a composition described herein has a pH between 6.0-7.0 (e.g., 6.6) and comprises a CD38-binding fusion protein at a concentration of 8-12 mg/mL (e.g., 10 mg/ml), histidine/histidine-HCl at a concentration of 40-60 mM (e.g., 50 mM), arginine-HCl at a concentration of 75-125 mM (e.g., 100 mM), sucrose at a concentration of 30-80 mg/ml (e.g., 50 mg/ml), and polysorbate 80 at a 0.1-0.3 mg/ml (e.g., 0.2 mg/ml).

In some embodiments, a composition comprising the CD38-binding fusion protein used in a method described herein comprises a CD38-binding fusion protein at a concentration that does not exceed 100 mg/ml. In some embodiments, a composition comprising the CD38-binding fusion protein used in a method described herein comprises a CD38-binding fusion protein at a concentration of 8-12 mg/ml. For example, a composition comprising the CD38-binding fusion protein used in a method described herein may comprise a CD38-binding fusion protein at a concentration of 8-12 mg/ml, 8-11.5 mg/ml, 8-11 mg/ml, 8-10.5 mg/ml, 8-10 mg/ml, 8-9.5 mg/ml, 8-9 mg/ml, 8-8.5 mg/ml, 8.5-12 mg/ml, 8.5-11.5 mg/ml, 8.5-11 mg/ml, 8.5-10.5 mg/ml, 8.5-10 mg/ml, 8.5-9.5 mg/ml, 8.5-9 mg/ml, 9-12 mg/ml, 9-11.5 mg/ml, 9-11 mg/ml, 9-10.5 mg/ml, 9-10 mg/ml, 9-9.5 mg/ml, 9.5-12 mg/ml, 9.5-11.5 mg/ml, 9.5-11 mg/ml, 9.5-10.5 mg/ml, 9.5-10 mg/ml, 10-12 mg/ml, 10-11.5 mg/ml, 10-11 mg/ml, 10-10.5 mg/ml, 10.5-12 mg/ml, 10.5-11.5 mg/ml, 10.5-11 mg/ml, 11-12 mg/ml, 11-11.5 mg/ml, or 11.5-12 mg/ml. In some embodiments, a composition comprising the CD38-binding fusion protein used in a method described herein comprises a CD38-binding fusion protein at a concentration of about 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, or 12 mg/ml. In some embodiments, a composition comprising the CD38-binding fusion protein used in a method described herein comprises a CD38-binding fusion protein at a concentration of about 10 mg/ml.

In some embodiments, a composition comprising the CD38-binding fusion protein used in a method described herein has a pH of 5.5-7.5. For example, a composition comprising the CD38-binding fusion protein used in a method described herein may have a pH of 5.5-7.5, 5.5-7, 5.5-6.5, 5.5-6, 6-7.5, 6-7, 6-6.5, 6.5-7.5, 6.5-7, or 7-7.5. In some embodiments, a composition described herein has a pH of about 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5. In some embodiments, a composition comprising the CD38-binding fusion protein used in a method described herein has a pH of about 6.0-7.0 (e.g., 6.0-7.0, 6.2-7, 6.3-6.9, 6.4-6.8, or 6.5-6.7). In some embodiments, a composition comprising the CD38-binding fusion protein used in a method described herein has a pH of about 6.6.

A composition comprising the CD38-binding fusion protein used in a method as described herein further comprises a buffer (e.g., a histidine/histidine-HCl buffer), a tonicity agent (e.g., arginine-HCl), a stabilizer (e.g., sucrose), and a surfactant (e.g., polysorbate such as polysorbate 80).

In some embodiments, a composition comprising the CD38-binding fusion protein used in a method described herein comprises a buffer comprising histidine and histidine-HCl. In some embodiments, the histidine and histidine-HCl balance results in a final histidine concentration in the composition of 10-120 mM (e.g., 10-120 mM, 20-110 mM, 30-100 mM, 40-90 mM, 50-80 mM, or 60-70 mM). In some embodiments, the histidine and histidine-HCl balance results in a final histidine concentration in the composition of 12.5-107.5 mM. In some embodiments, the histidine and histidine-HCl balance results in a final histidine concentration in the composition of 15-50 mM (e.g., about 15 mM, about 20 mM, about 25 mM, about 30 mM, about 35 mM, about 40 mM, about 45 mM, or about 50 mM). In some embodiments, the histidine and histidine-HCl balance results in a final histidine concentration in the composition of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mM of histidine.

The relative amount of histidine and histidine-HCl may be adjusted, e.g., to achieve a desired pH, while maintaining the histidine concentration in the composition, as described herein. In some embodiments, the histidine and histidine-HCl balance results in a final histidine concentration in the composition of about 15 mM (e.g., when the composition comprises a buffer comprising histidine at a concentration of 7.5 mM and histidine-HCl at a concentration of 7.5 mM). In some embodiments, the histidine and histidine-HCl balance results in a final histidine concentration in the composition of about 50 mM (e.g., when the composition comprises a buffer comprising histidine at a concentration of 40 mM and histidine-HCl at a concentration of 10 mM).

In some embodiments, a composition comprising the CD38-binding fusion protein used in a method described herein comprises a tonicity agent comprising arginine-HCl. In some embodiments, a composition comprising the CD38-binding fusion protein used in a method described herein comprises arginine-HCl at a concentration of 50-125 mM (e.g., 50-125 mM, 60-120 mM, 70-110 mM, or 80-100 mM, 75-125 mM, 95-105 mM, or 97.5-102.5 mM). In some embodiments, a composition comprising the CD38-binding fusion protein used in a method described herein comprises arginine-HCl at a concentration of about 50 mM, 55 mM, 60 mM, 65 mM, 70 mM, 75 mM, 80 mM, 85 mM, 90 mM, 95 mM, 100 mM, 105 mM, 110 mM, 115 mM, or 120 mM. In some embodiments, a composition comprising the CD38-binding fusion protein used in a method described herein comprises arginine-HCl at a concentration of about 100 mM.

In some embodiments, a composition comprising the CD38-binding fusion protein used in a method described herein comprises a stabilizer. In some embodiments, the stabilizer is a sugar. In some embodiments, the stabilizer is sucrose. In some embodiments, a composition comprising the CD38-binding fusion protein used in a method described herein comprises sucrose at a concentration of 3-10% w/v (equivalent to 30-100 mg/ml). For example, a composition described herein may comprise sucrose at a concentration of 3-10% w/v, 3-9% w/v, 3-8% w/v, 3-7% w/v, 3-6% w/v, 3-5% w/v, 3-4% w/v, 3-10% w/v, 3-9% w/v, 3-8% w/v, 3-7% w/v, 3-6% w/v, 3-5% w/v, 3-4% w/v, 4-10% w/v, 4-9% w/v, 4-8% w/v, 4-7% w/v, 4-6% w/v, 4-5% w/v, 5-10% w/v, 5-9% w/v, 5-8% w/v, 5-7% w/v, 5-6% w/v, 6-10% w/v, 6-9% w/v, 6-8% w/v, 5-7% w/v, 7-10% w/v, 7-9% w/v, 7-8% w/v, 8-10% w/v, 8-9% w/v, or 9-10% w/v (equivalent to 30-100 mg/ml, 30-90 mg/ml, 30-80 mg/ml, 30-70 mg/ml, 30-60 mg/ml, 30-50 mg/ml, 30-40 mg/ml, 40-100 mg/ml, 40-90 mg/ml, 40-80 mg/ml, 40-70 mg/ml, 40-60 mg/ml, 40-50 mg/ml, 50-100 mg/ml, 50-90 mg/ml, 50-80 mg/ml, 50-70 mg/ml, 50-60 mg/ml, 60-100 mg/ml, 60-90 mg/ml, 60-80 mg/ml, 60-70 mg/ml, 70-100 mg/ml, 70-90 mg/ml, 60-80 mg/ml, 80-100 mg/ml, 80-90 mg/ml, or 90-100 mg/ml, respectively).

In some embodiments, a composition comprising the CD38-binding fusion protein used in a method described herein comprises sucrose at a concentration of about 3% w/v (equivalent to 30 mg/mL), 3.5% w/v (equivalent to 35 mg/mL), 4% w/v (equivalent to 40 mg/mL), 4.5% w/v (equivalent to 45 mg/mL), 5% w/v (equivalent to 50 mg/mL), 5.5% w/v (equivalent to 55 mg/mL), 6% w/v (equivalent to 60 mg/mL), 6.5% w/v (equivalent to 65 mg/mL), 7% w/v (equivalent to 70 mg/mL), 7.5% w/v (equivalent to 75 mg/mL), 8% w/v (equivalent to 80 mg/mL), 8.5% w/v (equivalent to 85 mg/mL), 9w/v (equivalent to 90 mg/mL), 9.5% w/v (equivalent to 95 mg/mL), or 10% w/v (equivalent to 100 mg/ml). In some embodiments, a composition comprising the CD38-binding fusion protein used in a method described herein comprises sucrose at a concentration of about 4%-8% w/v (equivalent to 40-80 mg/mL). In some embodiments, a composition comprising the CD38-binding fusion protein used in a method described herein comprises sucrose at a concentration of about 4%-7% w/v (equivalent to 40-70 mg/mL). In some embodiments, a composition comprising the CD38-binding fusion protein used in a method described herein comprises sucrose at a concentration of about 4%-6% w/v (equivalent to 40-60 mg/mL). In some embodiments, a composition comprising the CD38-binding fusion protein used in a method described herein comprises sucrose at a concentration of about 4.5%-5.5% w/v (equivalent to 45-55 mg/mL). In some embodiments, a composition comprising the CD38-binding fusion protein used in a method described herein comprises sucrose at a concentration of about 4% w/v, 5% w/v, 6% w/v, 7% w/v, or 8% w/v (equivalent to 40 mg/ml, 50 mg/ml, 60 mg/ml, 70 mg/ml or 80 mg/ml, respectively). In some embodiments, a composition comprising the CD38-binding fusion protein used in a method described herein comprises sucrose at a concentration of about 5% w/v (equivalent to 50 mg/ml).

In some embodiments, a composition comprising the CD38-binding fusion protein used in a method described herein comprises a surfactant. In some embodiments, the surfactant is a polysorbate. In some embodiments, the surfactant is a polysorbate 80 (PS80). In some embodiments, a composition comprising the CD38-binding fusion protein used in a method described herein comprises PS80 at a concentration of 0.005-0.03% w/v (equivalent to 0.05-0.3 mg/ml). For example, a composition comprising the CD38-binding fusion protein used in a method described herein may comprise PS80 at a concentration of 0.005-0.03% w/v, 0.005-0.025% w/v, 0.005-0.02% w/v, 0.005-0.015% w/v, 0.005-0.01% w/v, 0.01-0.03% w/v, 0.01-0.025% w/v, 0.01-0.02% w/v, 0.01-0.015% w/v, 0.015-0.03% w/v, 0.015-0.025% w/v, 0.015-0.02% w/v, 0.02-0.03% w/v, 0.02-0.025% w/v, 0.02-0.03% w/v, 0.02-0.025% w/v, or 0.025-0.03% w/v (equivalent to 0.05-0.3 mg/ml, 0.05-0.25 mg/ml, 0.05-0.2 mg/ml, 0.05-0.15 mg/ml, 0.05-0.1 mg/ml, 0.1-0.3 mg/ml, 0.1-0.25 mg/ml, 0.1-0.2 mg/ml, 0.1-0.15 mg/ml, 0.15-0.3 mg/ml, 0.15-0.25 mg/ml, 0.15-0.2 mg/ml, 0.2-0.3 mg/ml, 0.2-0.25 mg/ml, or 0.25-0.3 mg/ml, respectively). In some embodiments, a composition comprising the CD38-binding fusion protein used in a method described herein comprises PS80 at a concentration of about 0.007% w/v (equivalent to 0.07 mg/mL), 0.008% w/v (equivalent to 0.08 mg/mL), 0.009% w/v (equivalent to 0.09 mg/mL), 0.01% w/v (equivalent to 0.1 mg/mL), 0.011% w/v (equivalent to 0.11 mg/mL), 0.012% w/v (equivalent to 0.12 mg/mL), 0.013% w/v (equivalent to 0.13 mg/mL), 0.014% w/v (equivalent to 0.14 mg/mL), 0.015% w/v (equivalent to 0.15 mg/mL), 0.016% w/v (equivalent to 0.16 mg/mL), 0.017% w/v (equivalent to 0.17 mg/mL), 0.018% w/v (equivalent to 0.18 mg/mL), 0.019w/v (equivalent to 0.19 mg/mL), or 0.02% w/v (equivalent to 0.2 mg/mL). In some embodiments, a composition described herein comprises PS80 at a concentration of about 0.01%-0.03% w/v (equivalent to 0.1-0.3 mg/mL). In some embodiments, a composition comprising the CD38-binding fusion protein used in a method described herein comprises PS80 at a concentration of about 0.015%-0.025% w/v (equivalent to 0.15-0.25 mg/mL). In some embodiments, a composition comprising the CD38-binding fusion protein used in a method described herein comprises PS80 at a concentration of about 0.02% w/v (equivalent to 0.2 mg/ml).

In some embodiments, a composition comprising the CD38-binding fusion protein used in a method described herein comprises a CD38-binding fusion protein (e.g., a CD38-binding fusion protein as provided in Table 1) at a concentration of 8.5-11.5 mg/ml (e.g., 10 mg/ml), histidine (e.g., composed of histidine and histidine-HCl) at a concentration of 15-60 mM (e.g., 15 mM, 20 mM, 30 mM, 40 mM, or 50 mM), arginine-HCl at a concentration of 80-120 mM (e.g., 100 mM), sucrose at a concentration of 3-8% w/v (e.g., 5% w/v), and PS80 at a concentration of 0.01-0.03% w/v (e.g., 0.02% w/v), and wherein the composition is at a pH of 5.5-7.5 (e.g., 5.5, 6, 6.5, or 6.6). In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition comprising the CD38-binding fusion protein used in a method described herein comprises a CD38-binding fusion protein (e.g., a CD38-binding fusion protein as provided in Table 1) at a concentration of 10 mg/ml, histidine (e.g., composed of histidine and histidine-HCl) at a concentration of 50 mM, arginine-HCl at a concentration of 100 mM, sucrose at a concentration of 5% w/v, and PS80 at a concentration of 0.02% w/v, and wherein the composition is at a pH of 6.6. In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition comprising the CD38-binding fusion protein used in a method described herein comprises a CD38-binding fusion protein (e.g., a CD38-binding fusion protein as provided in Table 1) at a concentration of 10 mg/ml, histidine (e.g., composed of histidine and histidine-HCl) at a concentration of 15 mM, arginine-HCl at a concentration of 100 mM, sucrose at a concentration of 5% w/v, and PS80 at a concentration of 0.02% w/v, and wherein the composition is at a pH of 6. In some embodiments, the CD38-binding fusion protein comprises a heavy chain comprising the amino acid sequence of SEQ ID NO: 13 and a light chain comprising the amino acid sequence of SEQ ID NO: 10.

In some embodiments, a composition comprising the CD38-binding fusion protein used in a method described herein comprises a CD38-binding fusion protein (e.g., a CD38-binding fusion protein as provided in Table 1) at a concentration of about 30-100 mg/ml, histidine (e.g., composed of histidine and histidine-HCl) at a concentration of about 50-75 mM (e.g., 50 mM), arginine-HCl at a concentration of about 75-150 mM (e.g., 100 mM), sucrose at a concentration of about 3-10% w/v (e.g., 5% w/v), and PS80 at a concentration of about 0.01-0.06% w/v (e.g., 0.02% w/v), and wherein the composition is at a pH of about 6.0-7.0 (e.g., 6.5-6.7). In some embodiments, the CD38-binding fusion protein comprises a heavy chain comprising the amino acid sequence of SEQ ID NO: 13 and a light chain comprising the amino acid sequence of SEQ ID NO: 10. In some embodiments, a composition comprising the CD38-binding fusion protein used in a method described herein is an aqueous solution.

In some embodiments, a composition described herein (e.g., in a form of aqueous solution or in lyophilized form) is stored in dosage unit form. In some embodiments, a lyophilized form of a composition described herein is stored for at least 2 months, at least 4 months, at least 6 months, at least 1 year, at least 2 years, or at least 3 years. In some embodiments, a composition described herein (e.g., in a form of aqueous solution or in lyophilized form) is stored frozen.

In some embodiments, a method described herein is effective in treating a cancer in a patient. Treating may include, for example, inhibiting or reducing proliferation of CD38-positive cells in the cancer and/or inducing apoptosis of CD38-positive cells in the cancer.

The terms “subject” and “patient” are used interchangeably and include any mammals, including companion and farm mammals, as well as rodents, including mice, rabbits, and rats, and other rodents. Non-human primates, such as Cynomolgus monkeys, are more preferred, and human beings are highly preferred. In some embodiments, the subject is a human. In some embodiments, the subject is a human adult (e.g., more than 18 years old, including 18 years old). In some embodiments, the subject is a non-adult human (e.g., less than 18 years old).

The terms “treatment”, “treating”, “treat”, and the like, refer to obtaining a desired pharmacologic and/or physiologic effect. The effect may be prophylactic in terms of completely or partially preventing a disease or symptom thereof or reducing the likelihood of a disease or symptom thereof and/or may be therapeutic in terms of a partial or complete cure for a disease and/or adverse effect attributable to the disease. “Treatment”, as used herein, covers any treatment of a disease in a mammal, particularly In a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition.

Any one of the methods described herein is suitable for treating cancer. In some embodiments, the cancer is a CD38-expressing cancer. In some embodiments, the cancer is not a CD38-expressing cancer. In some embodiments, the cancer is a solid tumor. In some embodiments, the cancer is a metastatic solid tumor. In some embodiments, the cancer is a CD38-expressing, metastatic solid tumor. Tumors that may be treated include, but are not limited to colorectal cancer, bile duct cancer, bone cancer, gastroesophageal cancer, pancreatic cancer, renal cancer, melanoma, anal cancer, uterine cancer, liver cancer, muscle cancer, breast cancer, bladder cancer, brain cancer, head and neck cancer, ovarian cancer, cervical cancer, prostate cancer, endometrial cancer, or stomach cancer. In some embodiments, the cancer is metastatic melanoma, metastatic non-small cell lung cancer, head and neck squamous cell carcinoma, esophageal squamous cell carcinoma, metastatic urothelial carcinoma, gastric or gastroesophageal junction (GEJ) adenocarcinoma, triple-negative breast cancer, high-risk non-muscle invasive bladder cancer, advanced urothelial bladder cancer, Merkel cell carcinoma, microsatellite instability-high cancer, and cutaneous squamous cell carcinoma. In some embodiments, the cancer is melanoma (e.g., advanced or metastatic melanoma). In some embodiments, the cancer is advanced non-small cell lung cancer. In some embodiments, the cancer is head and neck squamous cell cancer. In some embodiments, the cancer is high-risk non-muscle invasive bladder cancer. In some embodiments, the cancer is advanced urothelian bladder cancer. In some embodiments, the cancer is kidney cancer. In some embodiments, the cancer is advanced MSI-H/dMMR colorectal cancer. In some embodiments, the cancer is microsatellite instability-high cancer. In some embodiments, the cancer is high-risk early-stage triple negative breast cancer. In some embodiments, the cancer is advanced triple negative breast cancer. In some embodiments, the cancer is advanced gastric cancer. In some embodiments, the cancer is advanced cervical cancer. In some embodiments, the cancer is advanced MSI-H/dMMR endometrial cancer. In some embodiments, the cancer is advanced liver cancer. In some embodiments, the cancer is advanced Merkel cell carcinoma. In some embodiments, the cancer is advanced esophageal cancer. In some embodiments, the cancer is cutaneous squamous cell carcinoma. In some embodiments, the cancer is a lymphoma. In some embodiments, the lymphoma is a Hodgkin lymphoma (e.g., Nodular sclerosis Hodgkin lymphoma (NSCH), Mixed cellularity Hodgkin lymphoma (MCCHL), Lymphocyte-rich Hodgkin lymphoma, or Lymphocyte-depleted Hodgkin lymphoma). In some embodiments, the lymphoma is a non-Hodgkin lymphoma (e.g., T cell lymphoma, B cell lymphoma, NK cell lymphoma, or primary cutaneous lymphoma).

### EXAMPLES

### Example 1: An Open-Label, Dose Escalation Phase 1b Study to Evaluate the Safety, Tolerability, Pharmacokinetics, Pharmacodynamics and Antitumor Activity of a CD38-Binding Fusion Protein as a Single Agent and in Combination with Pembrolizumab in Adult Patients with Advanced or Metastatic Solid Tumors

### Background

CD38 is a multifunctional ectoenzyme involved in cell adhesion and transmembrane signaling. It is overexpressed in various tumor types and is believed to play a role in tumor cell migration and metastasis. CD38 is an approximately 45 kDa transmembrane glycoprotein expressed by immature hematopoietic cells, downregulated in mature cells, and re-expressed at higher levels by activated lymphocytes, such as T cells, B cells, dendritic cells, and natural killer (NK) cells.

Early bone marrow cells, which are crucial for long-term (sustained) marrow recovery, do not express CD38, but committed progenitor bone marrow cells, B cells in germinal centers, terminally differentiated plasma cells, and activated tonsils are CD38+. CD38 is expressed in hematologic precursor plasma cells of bone marrow where it functions in homing and apoptosis and is considered to be a marker of precursor cell commitment. CD38 is found throughout thymic development in thymus tissue. CD38 is expressed in germinal center B cells in the spleen and lymph nodes where it functions in rescue from apoptosis. CD38 is expressed in T, B, and NK cells, monocyte subsets, platelets, erythrocytes, and hematological precursor plasma cells where it interacts with endothelium. CD38 is expressed in the intraepithelial and lamina propria lymphocytes of the gut where it functions in mucosal immunity. CD38 is expressed in Purkinje cells and found in neurofibrillary tangles in the brain where it functions in memory processes. CD38 is expressed in the epithelial cells of the prostate. CD38 is expressed in R cells of the pancreas where it functions in insulin secretion. CD38 is expressed in osteoclasts of bone tissue where is functions in bone resorption. CD38 is expressed in retinal cells of the eye where it functions in vision processes. CD38 is expressed in the sarcolemma of smooth and striated muscle where it functions in muscle contraction. CD38 is also found in a soluble form in normal and pathological fluids.

The CD38-binding fusion protein evaluated in this study is a recombinant humanized immunoglobulin (Ig) G4 anti-CD38 monoclonal antibody fused to 2 attenuated interferon-alpha 2b (IFNα2b) moieties. The amino acid sequences of the CD38-binding fusion protein are provided in Table 1 (heavy chain of SEQ ID No: 13 and light chain of SEQ ID NO: 10). The CD38-binding fusion protein was produced by recombinant DNA technology in a mammalian cell expression system and was purified by a process that included specific viral inactivation and removal steps. The CD38 antibody portion of CD38-binding fusion protein directs the attenuated IFNα2b portion to CD38-expressing (CD38+) cells, thus achieving a high local concentration of IFNα2b at the surface of these target cells. On CD38-negative cells, the attenuation resulted is approximately 130,000-fold reduced potency compared with IFNα2b.

The CD38-binding fusion protein has a high binding affinity (dissociation constant [K_{D}]) for human and cynomolgus CD38, with a K_{D} of 168 pM and 1.25 nM, respectively. CD38-binding fusion protein potently inhibits proliferation of CD38+ multiple myeloma (MM) cells (half-maximal inhibitory concentration [IC50]19.9 pM), whereas potency on CD38-negative cells is approximately 2500-fold lower. The antibody portion of CD38-binding fusion protein is an IgG4 isotype (unlike the IgG1 isotype of daratumumab) and therefore had limited effector capacity to induce antibody-dependent cell-mediated cytotoxicity, antibody-dependent cellular phagocytosis, or complement activation against normal CD38+ cells. CD38-binding fusion protein does not modulate the adenosine diphosphate-ribosyl cyclase activity of CD38, unlike daratumumab.

A promising moiety to be conjugated to an anti-CD38 monoclonal antibody (mAb) is the cytokine interferon-alpha (IFNα), which is currently approved—although seldom used—for maintenance treatment for multiple myeloma (MM), metastatic melanoma, follicular lymphoma, or hairy cell leukemia. IFNα has direct inhibitory effects on some tumors and is a potent stimulator of both the innate and adaptive immune systems.

Systemic toxicity of IFN-α, however, precludes the use of the cytokine at therapeutically effective doses in the majority of patients. By reducing the binding affinity of IFNα for its IFNα receptor, CD38-binding fusion protein is expected to limit binding of attenuated IFNα to its receptor on non-CD38-\-expressing cells. In contrast, binding of CD38-binding fusion protein with high affinity via its CD38 targeting moieties is expected to increase the local concentration of attenuated IFNα on CD38+ target cells, thereby inducing desired on-target interferon (IFN) pathway activation.

Interferon-α2b (Intron A) by comparison has similar potency to CD38-binding fusion protein on CD38+ cells (IC₅₀ 12.3 pM) but on CD38-negative cells was approximately 130,000-fold more potent than CD38-binding fusion protein (EC₅₀ [half-maximal effective concentration]~0.37 pM).

Robust, complete antitumor responses in CD38-expressing, IFNα-sensitive xenograft models of MM following treatment with CD38-binding fusion protein containing a recombinant humanized immunoglobulin (Ig) G4 anti-CD38 monoclonal antibody fused to 2 attenuated interferon-alpha 2b (IFNα2b) moieties was previously observed. The antitumor activity was driven by direct antiproliferative activity of IFNα and indirect activation of innate immune cell subtypes including M1 macrophage and NK cells.

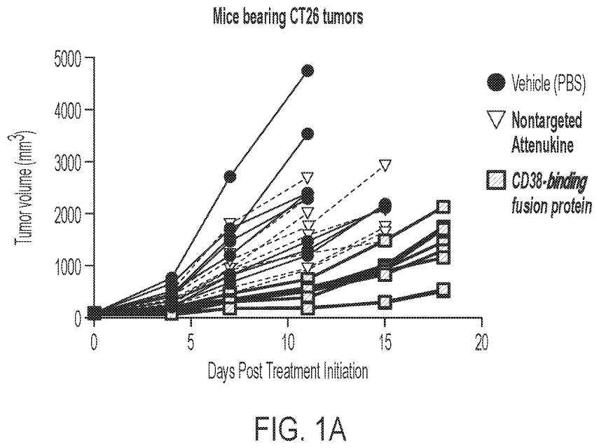

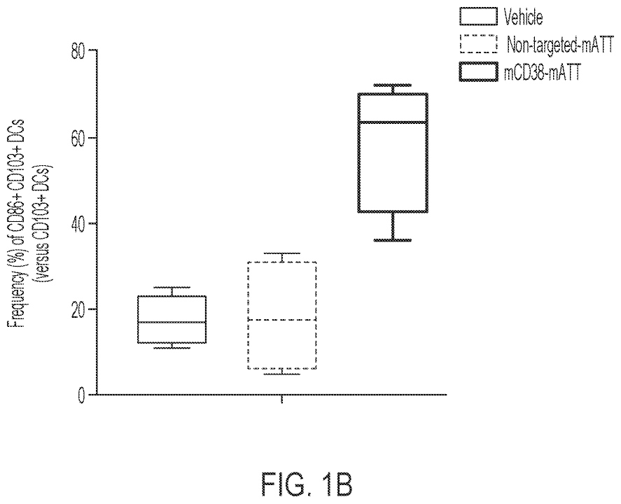

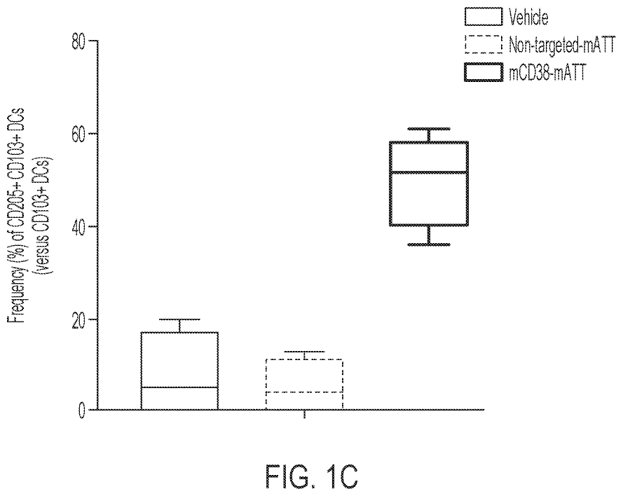

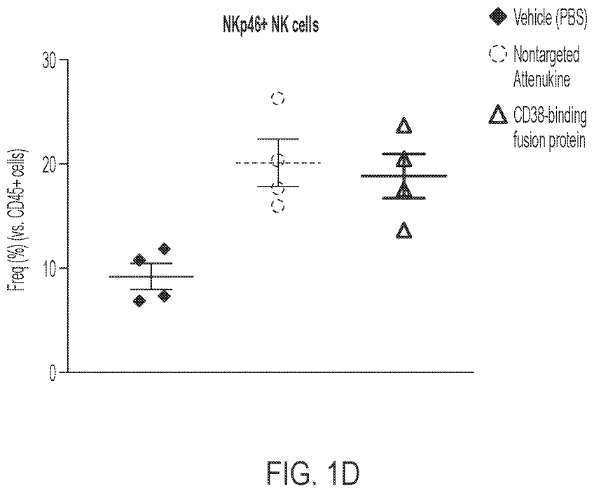

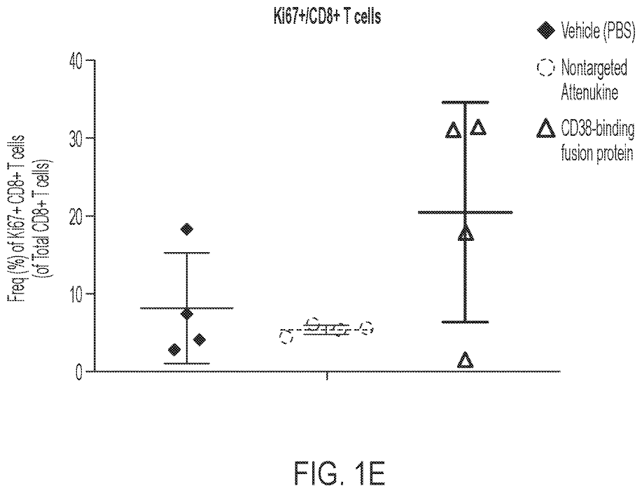

Further studies were conducted evaluating the activity of a murine cross-reactive surrogate of CD38-binding fusion protein (i.e., anti-mouse CD38-mIgG1-murine attenukine [mCD38-mATT]) in non-CD38-expressing, mildly IFNα sensitive murine tumor models in immunocompetent mice. The data show that the surrogate exhibited significant antitumor activity compared to the nontargeted-mATT control (FIG. 1A). In addition, pharmacodynamic analyses highlighted significant activation of dendritic cells (as indicated by increased expression of the surface activation markers CD86 (FIG. 1B) and CD205 (FIG. 1C). Additionally, administration of CD38-binding fusion protein increased expression of NKp46 in NK cells (FIG. 1D) and Ki67 in T cells (FIG. 1E). These results indicated CD38-binding fusion protein increased proliferative capacity of CD8+ T cells post-treatment with mCD38-mATT.

### Study Design and Results

An open-label, phase 1b/2 study was designed to evaluate the safety, tolerability, pharmacokinetics (PK), pharmacodynamics and antitumor response of CD38-binding fusion protein as a single agent (SA) and in combination with pembrolizumab in patients with advanced or metastatic solid tumors. In phase 1b, patients were enrolled with advanced/metastatic solid tumors that have no standard therapeutic option, are intolerant to those therapies, or have refused them.

Each patient had to meet all the following inclusion criteria to be enrolled in the study: 1) Adult male or female patients aged ≥18 years; 2) Eastern Cooperative Oncology Group (ECOG) performance status of 0-1; 3) Life expectancy >12 weeks according to investigator's judgment; 4) Phase 1b dose escalation: Eligible patients must have histologically confirmed advanced (locoregionally recurrent, not amenable to curative therapy) or metastatic solid tumors; 5) Measurable disease per mRECIST v1.1. At least 1 target lesion amenable for biopsy is required for enrollment in phase 1b. A minimum of 1 target lesion for response assessment is required for enrollment in phase 2. A separate lesion amenable for biopsy is required for enrollment in phase 2 for cohorts I and II post futility analysis and for all patients (safety lead-in and expansion) with subgroup III melanoma; 6) phase 1b dose escalation: patients with histologically confirmed advanced locally (locoregionally recurrent, not amenable to curative therapy) or metastatic solid tumors.

Patients meeting any of the following exclusion criteria were not enrolled in the study: 1) treatment with any SoC or investigational anticancer drug within 28 days or 5 half-lives before administration of CD38-binding fusion protein, whichever comes first (the washout period is 3 weeks for previous major surgery, 2 weeks for previous radical radiation, including chemoradiation and whole-brain radiation), and 5 days from last dose for focal radiation for symptomatic metastases; 2.) persistent toxicity from previous treatments that has not resolved to ≤CTCAE v.5 Grade 1 prior to administration of CD38-binding fusion protein, except for alopecia, Grade 2 neuropathy, and Grade 2 asthenia/fatigue; 3) history of any of the following ≤6 months before the first dose of CD38-binding fusion protein: New York Heart Association Grade III or IV congestive heart failure, unstable angina, myocardial infarction, unstable symptomatic ischemic heart disease, any ongoing symptomatic cardiac arrhythmias Grade >2, pulmonary embolism, symptomatic cerebrovascular events, or any other serious cardiac condition (e.g., symptomatic pericardial effusion or restrictive cardiomyopathy). Chronic, stable atrial fibrillation on stable anticoagulant therapy, including low molecular-weight heparin, is allowed; 4) baseline QTcF >480 msec (Grade ≥2), history of congenital long QT syndrome, or torsades de pointes; 5) history of immune-related AEs related to treatment with prior CPI that required treatment discontinuation (phase 2); 6) psychiatric illness/social circumstances that would limit compliance with study requirements and substantially increase the risk of AEs or has compromised ability to provide written informed consent; 7) history of uncontrolled brain metastasis or previously treated metastases receiving corticosteroid dose >20 mg/day of prednisone equivalent at the time of receiving the first dose of CD38-binding fusion protein (note: Patients with carcinomatosis meningitis or leptomeningeal disease were excluded, regardless of clinical stability); 8) patients with uveal (ocular) or mucosal melanoma (phase 2); 9) ongoing or active infection; 10) Known history of HIV infection or any other relevant congenital or acquired immunodeficiency; 11) Known hepatitis B (HBV) surface antigen seropositive or detectable hepatitis C infection viral load (note: patients with a positive HBV core antibody can be enrolled but must have an undetectable hepatitis B viral load); 12) autoimmune disease requiring systemic immunosuppressive therapy (patients with immune mediated endocrine deficiency from previous therapy with stable hormone replacement were exceptions); 13) history of severe allergic or anaphylactic reaction to recombinant proteins or excipients used in CD38-binding fusion protein or pembrolizumab formulation; 14) female patients who are lactating and/or breastfeeding or have a positive serum pregnancy test during the screening period.

The CD38-binding fusion protein single agent dose escalation phase was designed to determine the single agent recommended phase II dose (RP2D) and schedule of CD38-binding fusion protein for further testing. The single agent RP2D may be either the maximal tolerable dose (MTD) based on dose limiting toxicity (DLT) or a PAD pharmacologically active dose (PAD) defined by the PK/pharmacodynamic model or exposure-response (ER) analysis in place.

Adult patients with advanced/metastatic solid tumors received CD38-binding fusion protein IV on day 1 of a 21-day cycle (Q3W). Dose escalation started at 0.1 mg/kg and proceeded based on cycle 1 safety data via a Bayesian model with overdose control principles. Twenty-one pts were dosed in the escalation phase at 0.1 (n=3), 0.2 (n=3), 0.4 (n=3), 0.75 (n=3), 1.0 (n=3), and 1.5 mg/kg (n=6) Q3W. The median age of patients is 63 y (range 42-80). 57.1% of the patients are male. 71.4% of the patients have GI malignancies. The median prior lines of therapy is 3 (range 2-7).

A minimum of 3 patients were enrolled in the starting dose cohort of CD38-binding fusion protein 0.1 mg/kg administered Q3W. Patients were administered the CD38-binding fusion protein at a dose of 0.1, 0.2, 0.4, 0.75, or 1.5 mg/kg of the subject's weight on Cycle 1 Day 1 (C1D1; beginning of dose escalation phase (1b)). Patients received multiple cycles of treatments with 21 days in each cycle. Patients were assessed for DLTs until Cycle 2 Day 1 (C2D1) from a safety standpoint. If there were no DLTs in the first cohort of 3 evaluable patients dosed at 0.1 mg/kg, Bayesian Logistic Regression Model (BLRM) guided by the Escalation with Overdose Control (EWOC) principle was used in successive dose escalation cohorts to estimate the next dose level. More conservative dose escalation, evaluation of intermediate doses, and expansion of an existing dose level were permissible if such measures were needed for patient safety or for a better understanding of the dose-related toxicity, exposure, or pharmacodynamics. Approximately 30 patients were enrolled until either the MTD and/or PAD was identified.

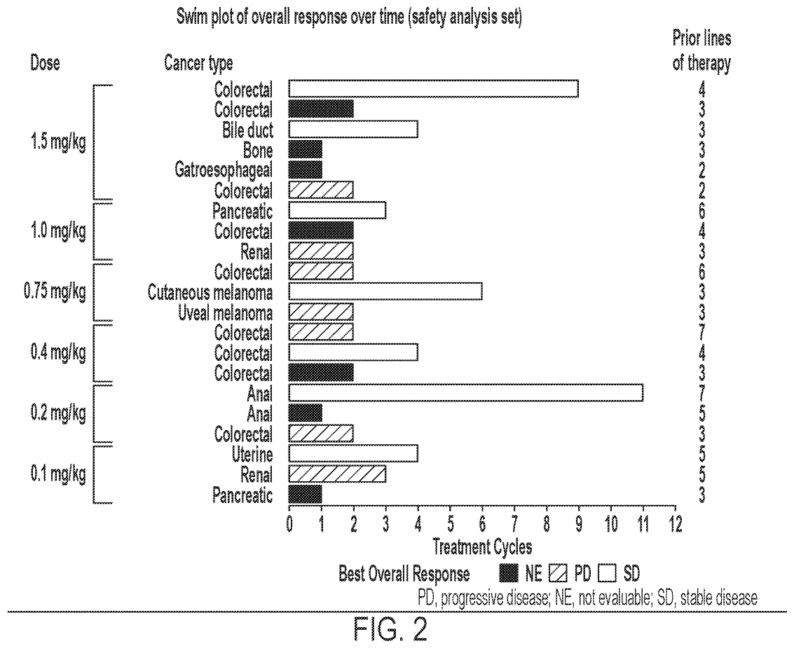

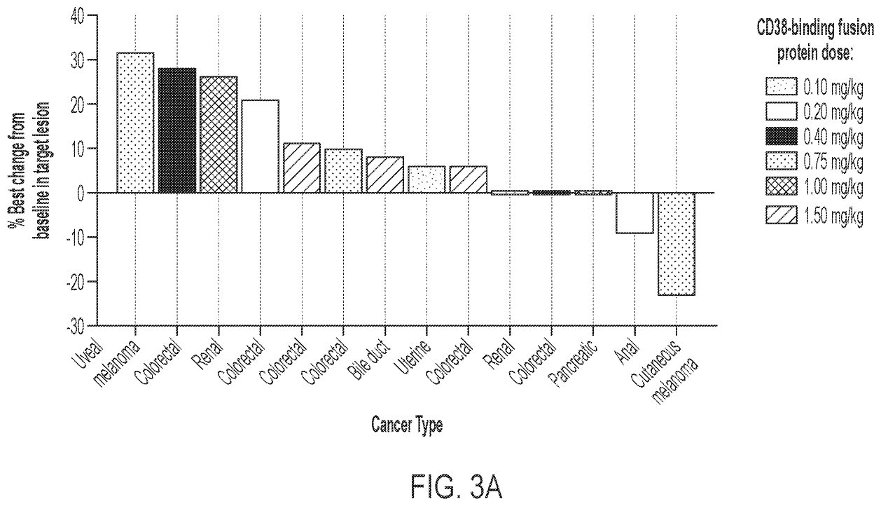

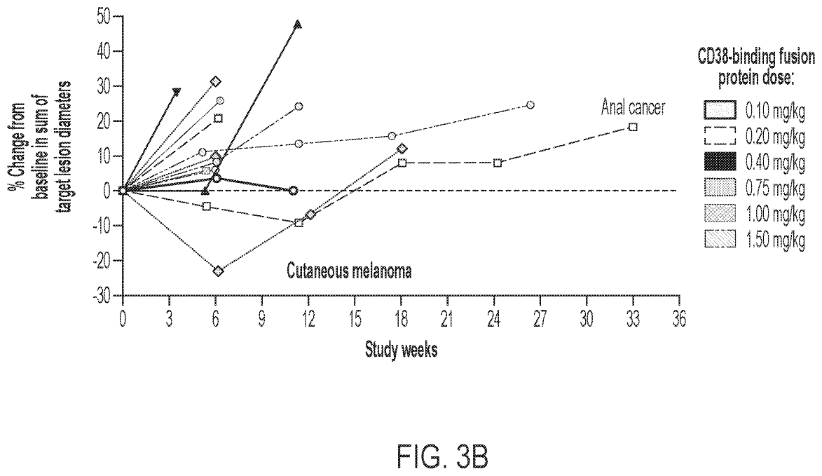

Patients diagnosed with various cancers were treated with the CD38-binding fusion protein for 1-11 cycles depending on the cancer type and disease progression (FIG. 2). Among the 14 response-evaluable patients, seven patients had best response of stable disease, including 1 with cutaneous melanoma who had 23% target lesion reduction (FIGS. 3A and 3B).

Two patients had dose-limiting toxicities in cycle 1 at 1.5 mg/kg; 1 patient with baseline bone infiltration had grade 4 thrombocytopenia and 1 patient had grade 3 confusion. Across all doses, patient received a median of 2 treatment cycles (range 1-11). CD38-binding fusion protein treatment-related adverse events (TRAEs) reported in 81% of patient included infusion-related reactions (52.4%), chills (47.6%), and nausea (33.3%). Grade ≥3 TRAEs reported in 42.9% of patients included neutropenia (9.5%) and hypertension (9.5%). Additionally, most patients had a transient decrease in platelet and neutrophil counts with rapid rebound to baseline at the end of each cycle. There was no clear relationship observed between the CD38-binding fusion protein dose and the degree of platelet and neutrophil count decrease.

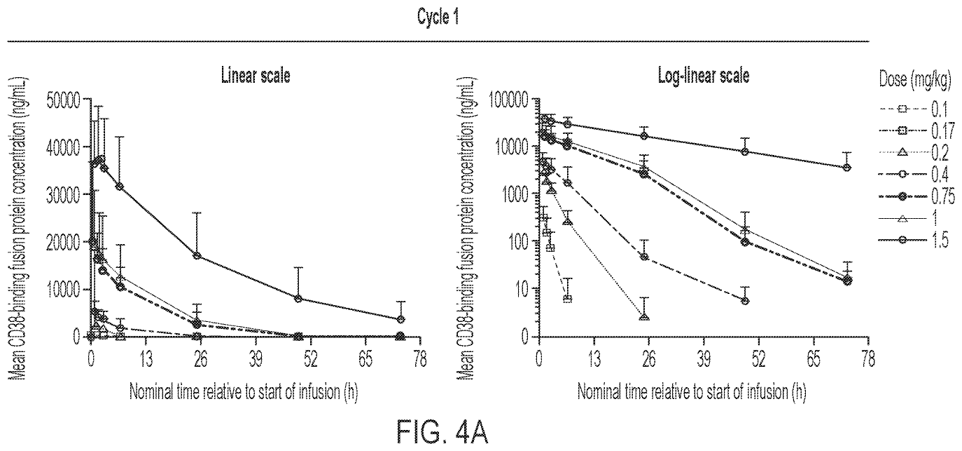

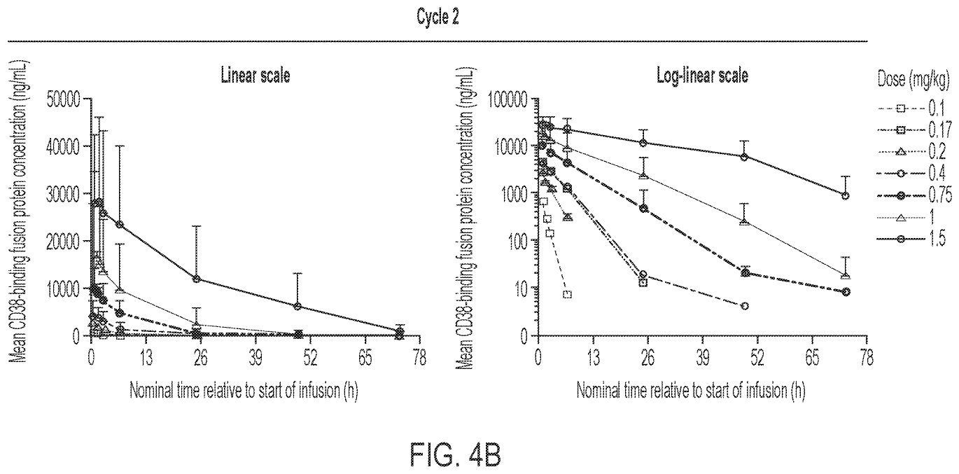

Pharmacokinetics and immunogenicity in patients as a result of CD38-binding fusion protein administration was determined. Serum concentrations of the CD38-binding fusion protein were generally measurable up to 6 h (0.1 mg/kg), 24 h (0.2 mg/kg), 48 h (0.4 mg/kg), and 72 h (0.75, 1.0, and 1.5 mg/kg) post end-of-infusion. AUC inter-patient variability was moderate to high in all dosage groups. There was a greater than dose proportional exposure increase in the dose range 0.1-1.5 mg/kg, with no exposure accumulation after Q3W dosing. The accumulation ratio in area under the plasma concentration (AUC) (i.e., time curve from time 0 to the last quantifiable concentration) was generally <1 across dose groups. In cycle 1, the geometric half-life ranged from approximately 1 hour (0.1 mg/kg) to 16 hours (1.5 mg/kg). The incidence rate of post-treatment with anti-drug antibody was 100% (FIGS. 4A and 4B).

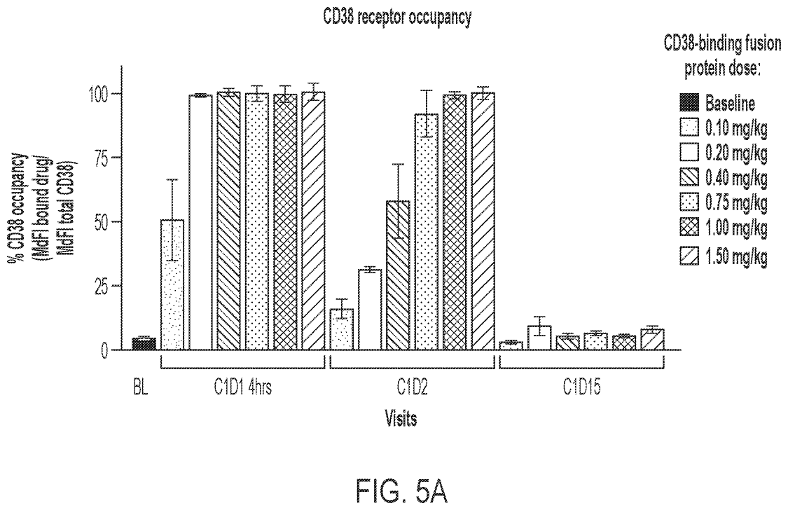

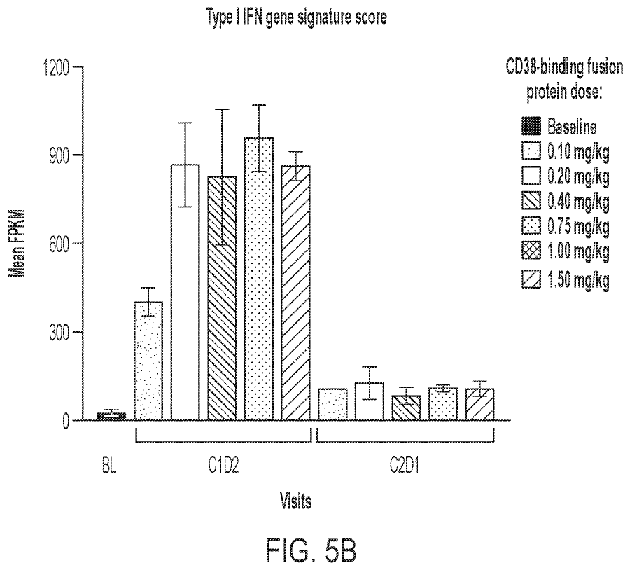

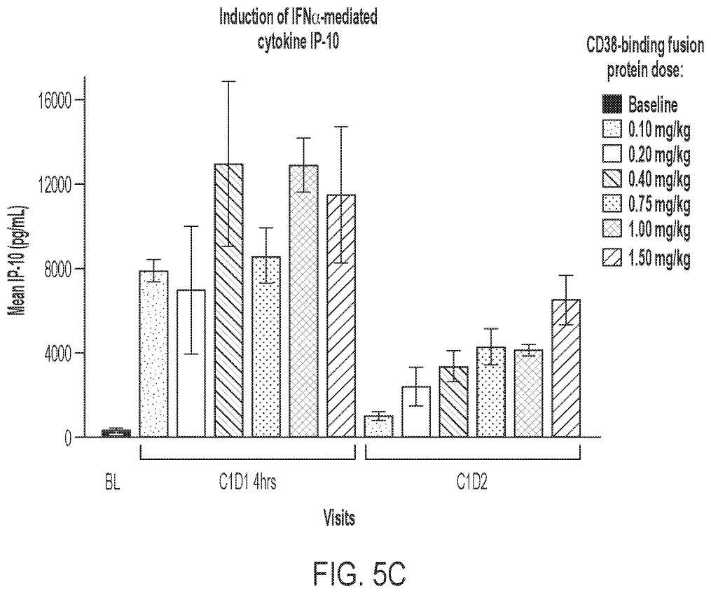

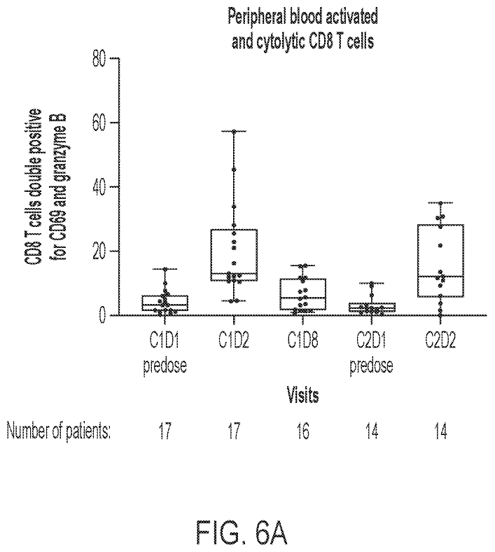

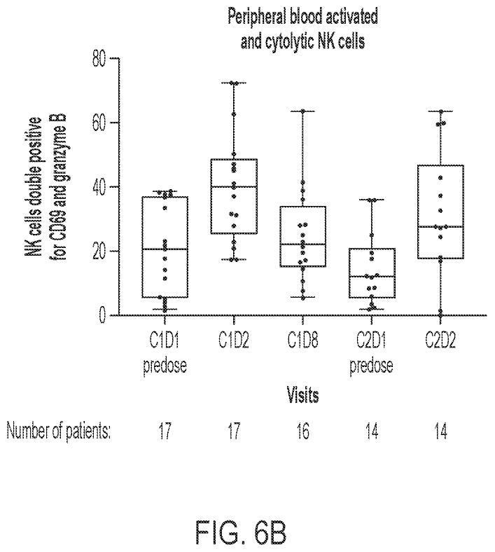

To further characterize the effect of CD38-binding fusion protein administration to patients, CD38 receptor occupancy (RO), interferon pathway activation, via a type I interferon gene signature score and systemic interferon alpha mediated cytokine concentrations, and innate and adaptive immune cell frequency and activation were analyzed. A dose-dependent saturation of CD38 receptor occupancy was observed (FIG. 5A). Additionally, administration of the CD38-binding fusion protein as a single agent resulted in induction of the type I IFN gene signature score (FIG. 5B). SA administrated also resulted in production of system IFNα-mediated cytokines and chemokines (FIG. 5C). Further analyses indicated that treatment with CD38-binding fusion protein resulted in expression of CD69 in CD8+ T cells (FIG. 6A) and expression of CD69 and granzyme B in NK cells (FIG. 6B). The pharmacodynamic data suggests that the peak IFN pathway modulation is achieved at ≥0.2 mg/kg in the peripheral blood with the duration of modulation increasing with dose.

Among the 14 response-evaluable patients, 7 had best response of stable disease, including 1 with cutaneous melanoma who had 21% target lesion reduction.

In conclusion, these results indicated CD38-binding fusion protein had a manageable safety profile in the dose range 0.10-1.50 mg/kg in patients with solid tumors. PD data suggested saturation of peak IFN pathway modulation at dose levels ≥0.2 mg/kg in the peripheral blood and that the duration of modulation increased with dose. Among the 14 response-evaluable pts, 7 had best response of stable disease, including 1 with cutaneous melanoma who had 21% target lesion reduction. The recommended single-agent phase 2 dose was determined to be 1.00 mg/kg Q3W, and the pharmacologic active dose was 0.10-1.50 mg/kg Q3W.

### Example 2: Phase 2 Dose Expansion

CD38-binding fusion protein had a manageable safety profile in the dose range 0.1-1.5 mg/kg in pts with solid tumors. The recommended phase 2 dose was determined as 1.0 mg/kg Q3W based on assessment of holistic data and is tested as a single agent and in combination with a checkpoint inhibitor (pembrolizumab) in selected tumor types. Pembrolizumab is a humanized immunoglobulin G4 (IgG4) mAb with a high specificity of binding to the PD-1 receptor, thus inhibiting its interaction with PD-L1 and programmed cell death protein 2 ligand (PD-L2). Preclinical data demonstrated the high affinity and potent receptor blocking activity for PD-1. Pembrolizumab has an acceptable clinical safety profile as an IV immunotherapy for advanced malignancies. Pembrolizumab is indicated for treatment across multiple indications.

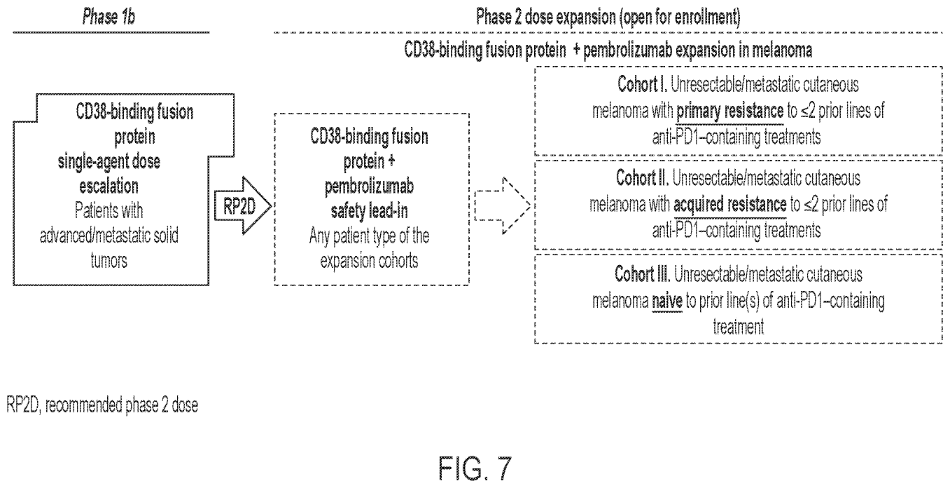

FIG. 7 shows the study design of the Phase 2 dose expansion phase. Phase 2 study begins with a safety-lead in period for all patients. After the safety-lead in period, patients are divided into three cohorts: (1) patients with unresectable/metastatic cutaneous melanoma with primary resistance to two or less prior lines of anti-PD1 containing treatments; (i) unresectable/metastatic cutaneous melanoma with acquired resistance to two or less prior lines of anti-PD1 containing treatments; and (3) unresectable/metastatic cutaneous melanoma naïve to prior lines of anti-PD1 containing treatments.

The single safety-lead in period evaluates safety and tolerability during the Cycle 1 DLT evaluation period of CD38-binding fusion protein with pembrolizumab. In the safety lead-in phase, if there are no DLTs in the first 3 patients during Cycle 1 of the safety lead-in period with CD38-binding fusion protein SA RP2D (1 mg/kg Q3W) in combination with pembrolizumab 400 mg Q6W, the patients in the three cohorts will be treated with the combination dose and regimen. If there is 1 DLT in the initial 3 patients in the safety-lead in period, an additional 3 patients are enrolled at the same CD38-binding fusion protein RP2D (1 mg/kg Q3W) in combination with pembrolizumab 400 mg Q6W. If there are ≥2 DLTs in the first 3 patients at the CD38-binding fusion protein SA RP2D (1 mg/kg Q3W), enrollment resumes at the next lower dose level of CD38-binding fusion protein RP2D (e.g., 0.75 mg/kg Q3W) in combination with pembrolizumab. Other approved pembrolizumab dosing regimens (e.g., 200 mg Q3W) could be tested in the safety-lead in period if 400 mg Q6W in combination with CD38-binding fusion protein at one dose lower than RP2D is intolerable.

### Example 3: a Phase 1/2 Open-Label Study to Investigate the Safety and Tolerability, Efficacy, Pharmacokinetics, and Immunogenicity of CD38-Binding Fusion Protein as a Single Agent in Patients with Relapsed Refractory Multiple Myeloma

Multiple myeloma (MM) is a plasma cell-derived malignancy characterized by bone lesions, hypercalcemia, anemia, and renal insufficiency. The 5-year survival rate of patients diagnosed with MM is approximately 45%. MM persists as a mostly incurable disease because of its highly complex and diverse cytogenetic and molecular abnormalities. There has been some improvement in the outcome for MM patients in the last decade with the discovery, development, and approval of proteasome inhibitors (PI) (e.g., bortezomib) and immunomodulatory imide drugs (IMiDs); however, patients who become refractory or are ineligible to receive bortezomib and IMiDs have a dismal prognosis.

Daratumumab, a CD38 antibody, is currently approved in many countries for the treatment of MM. Daratumumab was studied in patients who had received at least 3 prior lines of therapy, including a PI and an IMiD, or who were double-refractory to these agents. An objective response rate (ORR) of 29% was documented, including a 3% rate of complete response (CR)/stringent complete response (sCR). However, not all patients respond to daratumumab, and many patients eventually develop progressive disease (PD). Response to daratumumab therapy is significantly associated with CD38 expression levels on the tumor cells, and pretreatment levels of CD38 expression on MM cells were significantly higher in patients who achieved at least a partial response (PR) compared with patients who did not achieve a PR. In addition, CD38 expression in these patients was reduced in both bone marrow-localized and circulating MM cells following the first daratumumab infusion, and increased again following daratumumab discontinuation. Thus, there is still a need for development of novel targeted therapies that act specifically on the biology of the tumor cells, overcoming limitations related to the CD38 expression level.

### CD38-Binding Fusion Protein Targets

The tumor cell surface-expressed antigen CD38 is uniformly and highly expressed on MM cells and at lower levels on various lymphoid and myeloid cells and some solid organs. Being highly expressed on the myeloma cell surface and showing lower expression on normal cells makes CD38 an appropriate target for delivering drugs (cytokines, radioisotopes, and toxins) to receptor-expressing cells. A promising moiety to be conjugated to an anti-CD38 monoclonal antibody (mAb) is the cytokine interferon-alpha (IFN-α), which is currently used by clinicians as a potential maintenance treatment option for MM following primary treatment and autologous and allogeneic stem cell transplant (SCT). IFN-α has direct inhibitory effects on some tumors and is a potent stimulator of both the innate and adaptive immune systems. Systemic toxicity of IFN-α, however, precludes the use of the cytokine at therapeutically effective doses for the majority of patients. By reducing the binding affinity of IFN-α (KD) for its receptor, interferon alpha receptor, the CD38-binding fusion protein is expected to reduce binding of IFN-α to nontargeted, CD38-negative cells. The CD38-binding fusion protein evaluated in this study is a recombinant humanized immunoglobulin (Ig) G4 anti-CD38 monoclonal antibody fused to 2 attenuated interferon-alpha 2b (IFNα2b) moieties. The amino acid sequences of the CD38-binding fusion protein are provided in Table 1 (heavy chain of SEQ ID No: 13 and light chain of SEQ ID NO: 10).

Binding of CD38-binding fusion protein with high affinity via its CD38 targeting moieties is expected to increase the local concentration attenuated IFNα on these CD38+ target cells, thereby inducing desired on-target IFN pathway activation. In addition, IFN-α pathway activation induces up-regulation of CD38 messenger RNA and protein levels in malignant cells of patients with B cell chronic lymphocytic leukemia, suggesting that CD38-binding fusion protein may be able to increase CD38 target expression in MM and other CD38+ immune cells, thus overcoming the limitations seen with anti-CD38-depleting antibodies such as daratumumab. CD38-binding fusion protein increases CD38 expression on MM cells in vitro, which further supports this hypothesis. CD38 is a multifunctional ectoenzyme involved in cell adhesion and transmembrane signaling. It is over expressed in hematologic tumors, where it is believed to play a role in tumor cell migration and metastasis. CD38 has been reported to be highly expressed in 80% of MM patient-derived tumor cells. CD38 is an approximately 45 kDa transmembrane glycoprotein expressed by immature hematopoietic cells, down regulated in mature cells, and re-expressed at higher levels by activated lymphocytes such as T cells, B cells, dendritic cells, and natural killer (NK) cells. Early bone marrow cells, which are crucial for long-term (sustained) marrow recovery, do not express CD38 but committed progenitor bone marrow cells, B cells in germinal centers, terminally differentiated plasma cells, and activated tonsils are CD38+.

### Study Design and Initial Results

Described is a multicenter, open-label, phase 1/2 study to determine the safety, tolerability, and efficacy of CD38-binding fusion protein as an intravenous (IV) single agent and in combination with dexamethasone in patients with relapsed/refractory multiple myeloma (RRMM). Phase 1 provides an assessment of CD38-binding fusion protein antitumor activity against relapsed and refractory multiple myeloma (RRMM) with one or more single agent schedules. Phase 2 which includes the Part 2 expansion cohorts and the Part 3 extension cohorts, provides a more robust estimate of the safety profile, antimyeloma activity, PK, and pharmacodynamics and determines whether the maximum tolerated dose (MTD)/optimal biologic dose (OBD) is appropriate for future studies. The Part 2 expansion cohorts includes at least one combination cohort of CD38-binding fusion protein with dexamethasone using the same dose and schedule of CD38-binding fusion protein. The Part 3 extension component identifies a safe and efficacious dose level of CD38-binding fusion protein. In Part 3, patients are randomized 1:1 to receive CD38-binding fusion protein 120 or 240 mg once every 4 weeks (Q4W), stratified by their cytogenetics risk (high risk [(del17, t(4;14) and/or t(14;16)] vs standard risk) and myeloma type (IgA vs other).

Initial results in 2 patients indicated that 6 mg/kg CD38-binding fusion protein administered once every 4 weeks (Q4W schedule) exceeded the maximum tolerated dose (MTD). Results also indicated that administering 3 mg/kg of CD38-binding fusion protein on a Q4W schedule as a single agent was a MTD. Further, RRMM treatment efficacy in patients was observed at 1.5 mg/kg Q4W with no efficacy seen below 1.5 mg/kg Q4W.

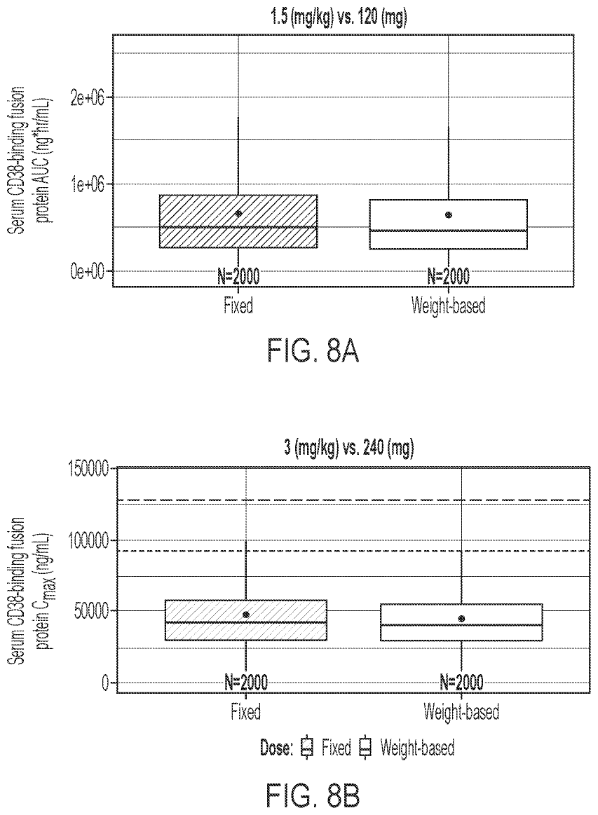

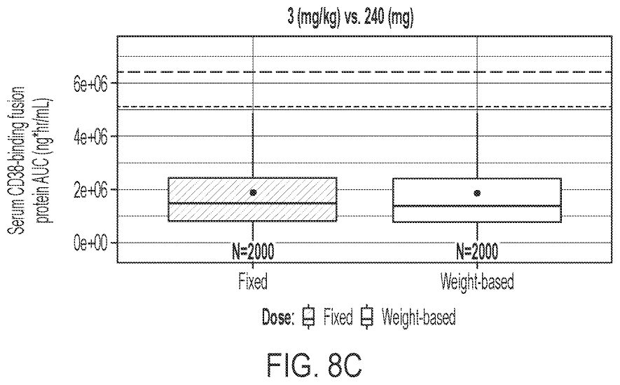

Fixed-dosing is also considered an appropriate dosing approach for further clinical development based on collective clinical findings and the benefits of fixed dosing due to its lower risk of dosing errors and substantial reduction of drug wastage. Additionally, computational analysis indicated that fixed-dosing and weight-adjusted dosing (e.g., mg/kg) would result in similar drug exposure in patients (FIG. 8A-8C). Accordingly, the 2 doses of 1.5 and 3 mg/kg CD38-binding fusion protein Q4W are translated into 2 fixed doses of 120 and 240 mg Q4W based on the median bodyweight of approximately 80 kg for the Part 3 evaluation.

Overall, this Example describes therapeutically effective and tolerated doses of CD38-binding fusion protein for treating RRMM.

What is claimed is:

1. A method of treating melanoma, the method comprising administering to a subject in need thereof a composition comprising a CD38-binding fusion protein, wherein the CD38-binding fusion protein comprises an anti-CD38 antibody fused to an attenuated interferon alpha-2b, wherein the CD38-binding fusion protein is administered at 0.1-1.5 mg/kg of the subject.

2. The method of claim 1, wherein the CD38-binding fusion protein is administered at 1 mg/kg of the subject.

3. The method of claim 1, wherein the CD38-binding fusion protein is administered at 0.75 mg/kg of the subject.

4. The method of claim 1, wherein the CD38-binding fusion protein is administered at 0.75-1.5 mg/kg of the subject.

5. A method of treating melanoma, the method comprising administering to a subject in need thereof a composition comprising 60-120 mg of a CD38-binding fusion protein, wherein the CD38-binding fusion protein comprises an anti-CD38 antibody fused to an attenuated interferon alpha-2b.

6. The method of claim 5, wherein 80 mg of the CD38-binding fusion protein is administered.

7. The method of claim 5, wherein 120 mg of the CD38-binding fusion protein is administered.

8. The method of any one of claims 1-7, wherein the CD38-binding fusion protein is administered once every three weeks.

9. The method of any one of claims 1-8, wherein the anti-CD38 antibody comprises a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, a light chain complementarity determining region 3 (CDR-L3) comprising the amino acid sequence of SEQ ID NO: 6.

10. The method of any one of claims 1-9, wherein the anti-CD38 antibody comprises a heavy chain variable region comprising the amino acid sequence of SEQ ID NO: 7 and a light chain variable region comprising the amino acid sequence of SEQ ID NO: 8.

11. The method of any one of claims 1-10, wherein the anti-CD38 antibody comprises a human IgG4 constant region.

12. The method of claim 11, wherein the human IgG4 constant region comprises a proline at position 228 according to the EU numbering system.

13. The method of claim 12, wherein the human IgG4 constant region further comprises a tyrosine at position 252, a threonine at position 254, and a glutamic acid at position 256 of the constant region according to the EU numbering system.

14. The method of any one of claims 1-13, wherein the anti-CD38 antibody comprises a heavy chain comprising the amino acid sequence of SEQ ID NO: 9 and a light chain comprising the amino acid sequence of SEQ ID NO: 10.

15. The method of any one of claims 1-14, wherein the attenuated interferon alpha-2b comprises T106A and A145D mutations relative to an interferon alpha-2b comprising the amino acid sequence of SEQ ID NO: 11.

16. The method of any one of claims 1-15, wherein the attenuated interferon alpha-2b comprises the amino acid sequence of SEQ ID NO: 12.

17. The method of any one of claims 14-16, wherein the attenuated interferon alpha-2b is fused to the C-terminus of the heavy chain.

18. The method of claim 17, wherein the CD38-binding fusion protein comprises a heavy chain comprising the amino acid sequence of SEQ ID NO: 13 and a light chain comprising the amino acid sequence of SEQ ID NO: 10.

19. The method of any one of claims 1-18, wherein the composition further comprises histidine, arginine, sucrose, and polysorbate 80 (PS80).

20. The method of claim 19, wherein in the composition, histidine is at a concentration of 50 mM, arginine is at a concentration of 100 mM, sucrose is at a concentration of 50 mg/ml, and PS80 is at a concentration of 0.2 mg/ml.

21. The method of any one of claims 1-20, wherein the composition is at a pH of 6.6.

22. The method of any one of claims 1-21, further comprising administering to the subject an immune checkpoint inhibitor.

23. The method of claim 22, wherein the immune checkpoint inhibitor is a PD-1 inhibitor.

24. The method of claim 23, wherein the PD-1 inhibitor comprises an anti-PD-1 antibody.

25. The method of claim 24, wherein the anti-PD1 antibody is pembrolizumab.

26. The method of claim 25, wherein 400 mg of pembrolizumab is administered once every six weeks.

27. The method of claim 25, wherein 200 mg of pembrolizumab is administered once every three weeks.

28. The method of any one of claims 1-27, wherein the melanoma expresses CD38.

29. The method of any one of claims 1-27, wherein the melanoma does not express CD38.

30. The method of any one of claims 1-29, wherein the subject is human.

31. A method of treating melanoma, the method comprising administering to a subject in need thereof a composition comprising a CD38-binding fusion protein, wherein the CD38-binding fusion protein comprises an anti-CD38 antibody fused to an attenuated interferon alpha-2b, wherein the CD38-binding fusion protein is administered at 0.1-1.5 mg/kg of the subject once every three weeks, and wherein the subject is receiving or has received treatment with an immune checkpoint inhibitor.

32. A method of treating melanoma, the method comprising administering to a subject in need thereof a composition comprising an immune checkpoint inhibitor, wherein the subject is receiving or has received treatment with a CD38-binding fusion protein, wherein the CD38-binding fusion protein comprises an anti-CD38 antibody fused to an attenuated interferon alpha-2b, wherein the CD38-binding fusion protein is administered at 0.1-1.5 mg/kg of the subject once every three weeks.

33. A method of treating melanoma, the method comprising administering to a subject in need thereof a composition comprising a CD38-binding fusion protein, wherein the CD38-binding fusion protein comprises an anti-CD38 antibody fused to an attenuated interferon alpha-2b, wherein 60-120 mg of the CD38-binding fusion protein is administered once every three weeks, and wherein the subject is receiving or has received treatment with an immune checkpoint inhibitor.

34. A method of treating melanoma, the method comprising administering to a subject in need thereof a composition comprising an immune checkpoint inhibitor, wherein the subject is receiving or has received treatment with a CD38-binding fusion protein, wherein the CD38-binding fusion protein comprises an anti-CD38 antibody fused to an attenuated interferon alpha-2b, wherein 60-120 mg of the CD38-binding fusion protein is administered once every three weeks.

35. A CD38-binding fusion protein for use in a method of treating melanoma, the method comprising administering to a subject in need thereof the CD38-binding fusion protein at 0.1-1.5 mg/kg of the subject once every three weeks, wherein the CD38-binding fusion protein comprises an anti-CD38 antibody fused to an attenuated interferon alpha-2b.

36. A CD38-binding fusion protein for use in a method of treating melanoma, the method comprising administering to a subject in need thereof 60-120 mg of the CD38-binding fusion protein once every three weeks, wherein the CD38-binding fusion protein comprises an anti-CD38 antibody fused to an attenuated interferon alpha-2b.

37. The method of any one of claims 31-34, or the CD38-binding fusion protein for use of claim 35 or claim 36, wherein the subject is further administered an immune checkpoint inhibitor.

38. The method or the CD38-binding fusion protein for use of claim 37, wherein the immune checkpoint inhibitor is pembrolizumab.

39. The method or the CD38-binding fusion protein for use of claim 38, wherein 400 mg of pembrolizumab is administered once every six weeks.

40. The method or the CD38-binding fusion protein for use of claim 38, wherein 200 mg of pembrolizumab is administered once every three weeks.

41. The method of any one of claims 31-34 and 37-40, or the CD38-binding fusion protein for use of any one of claims 35-40, wherein the CD38-binding fusion protein comprises a heavy chain comprising the amino acid sequence of SEQ ID NO: 13 and a light chain comprising the amino acid sequence of SEQ ID NO: 10.

42. The method of any one of claims 31-34 and 37-41, or the CD38-binding fusion protein for use of any one of claims 35-41, wherein the subject is human.

43. A method of treating multiple myeloma, the method comprising administering to a subject in need thereof a composition comprising a CD38-binding fusion protein, wherein the CD38-binding fusion protein comprises an anti-CD38 antibody fused to an attenuated interferon alpha-2b, wherein the CD38-binding fusion protein is administered at 1.5-6.0 mg/kg of the subject or wherein 120-240 mg of the CD38-binding fusion protein is administered to the subject.

44. The method of claim 43, wherein the CD38-binding fusion protein is administered at an amount between 1.5-6.0 mg/kg of the subject.

45. The method of claim 43, wherein the CD38-binding fusion protein is administered at an amount of 1.5 mg/kg of the subject.

46. The method of claim 43, wherein the CD38-binding fusion protein is administered at an amount of 3.0 mg/kg of the subject.

47. The method of claim 43, wherein the CD38-binding fusion protein is administered at an amount of 4.5 mg/kg of the subject.

48. The method of claim 43, wherein the CD38-binding fusion protein is administered at an amount of 6.0 mg/kg of the subject.

49. The method of claim 43, wherein 120-240 mg of the CD38-binding fusion protein is administered to the subject.

50. The method of any one of claim 49, wherein 120 mg of the CD38-binding fusion protein is administered to the subject.

51. The method of any one of claim 49, wherein 240 mg of the CD38-binding fusion protein is administered to the subject.

52. The method of any one of claims 43-51, wherein the CD38-binding fusion protein is administered once every four weeks.

53. The method of any one of claims 43-52, further comprising administering to the subject in need thereof pomalidomide, carfilzomib, daratumumab, lenzilumab, or bortezomib.

54. The method of any one of claims 43-53, wherein the anti-CD38 antibody comprises a heavy chain complementarity determining region 1 (CDR-H1) comprising the amino acid sequence of SEQ ID NO: 1, a heavy chain complementarity determining region 2 (CDR-H2) comprising the amino acid sequence of SEQ ID NO: 2, a heavy chain complementarity determining region 3 (CDR-H3) comprising the amino acid sequence of SEQ ID NO: 3, a light chain complementarity determining region 1 (CDR-L1) comprising the amino acid sequence of SEQ ID NO: 4, a light chain complementarity determining region 2 (CDR-L2) comprising the amino acid sequence of SEQ ID NO: 5, and a light chain complementarity determining region 3 (CDR-L3) comprising the amino acid sequence of SEQ ID NO: 6.

55. The method of any one of claims 43-54, wherein the attenuated interferon alpha-2b comprises the amino acid sequence of SEQ ID NO: 12.

56. The method of any one of claims 43-55, wherein the multiple myeloma expresses CD38.

57. The method of any one of claims 43-55, wherein the multiple myeloma does not express CD38.

58. The method of any one of claims 1-34, or 37-57, further comprising administering a corticosteroid.

59. The method of claim 58, wherein the corticosteroid is dexamethasone or methylprednisone.

60. The method of any one of claims 1-59, further comprising administering an anti-histamine.

61. The method of claim 60, wherein the antihistamine is montelukast or diphenhydramine.

62. The method claim 60 or claim 61, wherein the method comprises administering the antihistamine 12 hours before and 1 hour after administering the CD38-binding fusion protein to the subject for a first cycle of treatment and administering the antihistamine 1 hour before and 1 hour after administering the CD38-binding fusion protein to the subject for cycles of treatment after the first cycle of treatment.

63. The method of any one of claims 1-62, further comprising administering an analgesic.

64. The method of claim 63, wherein the analgesic comprises acetaminophen.

65. The method of claim 63, wherein the analgesic comprises an NSAID.

66. The method of any one of claims 1-34, or 37-42 or the CD38-binding fusion protein for use of claim 35 or claim 36, wherein the CD38-binding fusion protein is administered at an amount of 1.0 mg/kg of the subject.
